(19) United States
(12) Patent Application Publication
(10) Pub. No.: US 2019/0367790 A1
(43) Pub. Date: Dec. 5, 2019

(54) HEAT STORAGE MEDIUM, COOLING PACK, LOGISTICS PACKAGE, AND COOLING UNIT

(71) Applicant: SHARP KABUSHIKI KAISHA, Sakai City, Osaka (JP)

(72) Inventors: SATORU MOTONAMI, Sakai City (JP); YUKA UTSUMI, Sakai City (JP); HWISIM HWANG, Sakai City (JP); DAISUKE SHINOZAKI, Sakai City (JP); MASAKAZU KAMURA, Sakai City (JP)

(21) Appl. No.: 16/462,735

(22) PCT Filed: Nov. 22, 2017
(86) PCT No.: PCT/JP2017/042018

§ 371 Date: May 21, 2019

(30) Foreign Application Priority Data
Nov. 22, 2016 (JP) 2016-227098

(51) Int. Cl. C09K 5/06; C07C 19/075; F25D 3/08

(52) U.S. Cl. CPC C09K 5/06; C07C 19/075; F25D 3/08; F25D 2303/085; A61F 2007/108; F25D 2303/0841; F25D 2303/0844; F25D 2303/0845; F25D 2303/08222

(57) ABSTRACT

It is an object to provide a heat storage medium capable of maintaining the latent heat capacity even if a supercooling inhibitor is added. A heat storage medium according to an aspect of the present invention is a heat storage medium which undergoes a phase change at a predetermined temperature and contains water, a main agent made of a quaternary ammonium salt forming a semi-clathrate hydrate, a pH adjustor maintaining alkalinity, and a nucleating agent generating cations exhibiting positive hydration. The heat storage medium separates into a first liquid layer containing the main agent and a second liquid layer containing the nucleating agent in an environment with a temperature exceeding the phase change temperature.

### TECHNICAL FIELD

The present invention relates to a heat storage medium which undergoes a phase change at a predetermined temperature, a cooling pack, a logistics package, and a cooling unit.

### BACKGROUND ART

Clathrate hydrates (clathrate hydrates), particularly semi-clathrate hydrates (semi-clathrate hydrates), are crystallized by cooling aqueous solutions of main agents to or below the hydrate formation temperature. Since heat energy, which is usable as latent heat, is stored in crystals thereof, the clathrate hydrates have been hitherto used as heat storage mediums or components thereof.

Especially, hydrates of quaternary ammonium salts that are typical examples of semi-clathrate hydrates containing non-gas as a guest compound are formed at atmospheric pressure, have a large amount of heat energy (heat storage capacity) upon crystallization, and are non-flammable unlike paraffins. Thus, the quaternary ammonium salt hydrates are easy to handle and are attracting attention as means alternative to ice heat storage tanks for building air conditioning.

In particular, the latent heat energy of a semi-clathrate hydrate containing tetra-normal-butylammonium bromide or tri-normal-butyl-normal-pentylammonium bromide as a guest is obtained at higher temperature as compared to ice. Therefore, semi-clathrate hydrates have been increasingly used for heat storage tanks more efficient than ice heat storage tanks and heat transport media.

However, the temperature at which a semi-clathrate hydrate is formed, that is, the solidification temperature at which a liquid phase crystallizes into a solid phase is strongly affected by the supercooling phenomenon of water, the difference between the solidification temperature and the melting temperature which is the temperature at which latent heat is obtained is very large, and the semi-clathrate hydrate is difficult to handle. Therefore, supercooling inhibitors such as minerals have been hitherto used for the purpose of reducing the effect of supercooling.

Patent Literature 1 discloses a technique for charging a specific additive into an aqueous solution of raw materials. In this technique, disodium hydrogen phosphate and a thickening agent are added to 33 wt % tetrabutylammonium bromide (TBAB).

### CITATION LIST

### Patent Literature

PTL 1: Japanese Unexamined Patent Application Publication No. 2013-060603

### SUMMARY OF INVENTION

### Technical Problem

However, in the technique described in Patent Literature 1, freezing is unstable in a general refrigerator, moisture remains even if freezing is performed at 3° C., and complete freezing does not occur in some cases. Furthermore, adding a supercooling inhibitor and the thickening agent reduces the latent heat capacity.

The present invention has been made in view of the above circumstances and has an object to provide a heat storage medium capable of maintaining the latent heat capacity even if a supercooling inhibitor is added, a cooling pack, a logistics package, and a cooling unit.

### Solution to Problem

In order to achieve the above object, an aspect of the present invention has provided a means below. That is, a heat storage medium according to the present invention is a heat storage medium which undergoes a phase change at a predetermined temperature and contains water, a main agent made of a quaternary ammonium salt forming a semi-clathrate hydrate, a pH adjustor maintaining alkalinity, and a nucleating agent generating cations exhibiting positive hydration. The heat storage medium separates into a first liquid layer containing the main agent and a second liquid layer containing the nucleating agent in an environment with a temperature exceeding the phase change temperature.

### Advantageous Effects of Invention

According to an aspect of the present invention, a heat storage medium separates into a first liquid layer containing a main agent and a second liquid layer containing a nucleating agent separate in an environment with a temperature exceeding the phase change temperature; hence, the latent heat capacity is not reduced but is maintained regardless of adding a supercooling prevention agent. This enables a large amount of heat energy to be used. Furthermore, a separated portion serves as a nucleating agent and can be frozen in a general refrigerator.

### BRIEF DESCRIPTION OF DRAWINGS

FIG. 1 is a graph showing that the latent heat capacity depends on the concentration of TBAB.

FIG. 2 is a graph showing results of DSC experiments of Examples 1-3 and Comparative Examples 1-7.

FIG. 3 is a graph showing results of DSC experiments of Comparative Examples 1-5.

FIG. 4 is a graph showing results of DSC experiments of Examples 1-3 and Comparative Examples 6-7.

FIG. 5 is a graph showing comparative results of Examples 2 and 3.

FIG. 6 is a graph showing comparative results of Examples 1 and 3.

FIG. 7 is a graph showing results of an XRD experiment.

FIG. 8 is a graph showing results of XRD experiments on Examples 10 and 11.

FIG. 9 is a graph showing the relationship between the time and temperature of Example 12, Example 13, and Comparative Example 10.

FIG. 10A is a graph showing the relationship between the time and temperature of Example 12, Example 13, and Comparative Example 10.

FIG. 10B is a table showing the composition and holding time proportion of Example 12, Example 13, and Comparative Example 10.

FIG. 11 is an illustration showing results obtained by measuring “pH, refractive index, and Brix value” in a “TBAB38 wt %+P2.5%+C2%” system.

FIG. 12 shows results obtained by subjecting each of the first liquid layer 10 and the second liquid layer 20 to a DSC experiment in a “TBAB38 wt %+P2.5%+C2%” system.

FIG. 13 is an illustration showing results obtained by measuring the specific gravity for an Example 1 (TBAB38 wt %+PC) system.

FIG. 14A is an illustration showing the outline of an aqueous solution containing a main agent only.

FIG. 14B is an illustration showing the outline of an aqueous solution obtained by adding a supercooling inhibitor to a main agent.

FIG. 15 is a graph showing the dependence of the latent heat capacity on the concentration of tetrabutylammonium chloride (TBAC).

FIG. 16 is a graph showing the dependence of the latent heat capacity on the concentration of tetrabutylammonium nitrate (TBAN).

FIG. 17 is a graph showing the dependence of the latent heat capacity on the concentration of tetrabutylammonium fluoride (TBAF).

FIG. 18 is a graph showing results of an experiment for measuring the change in temperature of aqueous solutions of TBAC.

FIG. 19 is a graph showing results of an experiment for measuring the change in temperature of aqueous solutions of TBAN.

FIG. 20 is a graph showing results of an experiment for measuring the change in temperature of aqueous solutions of TBAF.

FIG. 21 is a sectional view of a cooling pack according to Example 23.

FIG. 22A is a conceptual view showing a step for manufacturing a cooling pack according to this example.

FIG. 22B is a conceptual view showing a step for manufacturing the cooling pack according to this example.

FIG. 22C is a conceptual view showing a step for manufacturing the cooling pack according to this example.

FIG. 23A is a sectional view of a logistics package according to this example.

FIG. 23B is a sectional view of a variation of the logistics package according to this example.

FIG. 23C is a sectional view of a variation of the logistics package according to this example.

FIG. 23D is a conceptual view showing the usage state of the cooling pack and logistics package according to this example.

FIG. 24 is a schematic view showing an example of a cooling unit according to this example.

FIG. 25 is a schematic view showing an example of the cooling unit according to this example.

FIG. 26 is a conceptual view showing an example of the usage state of the cooling unit according to this example.

FIG. 27 is a sectional view showing an example of the usage state of the cooling unit according to this example.

FIG. 28 is a perspective view showing the outline of a cooling pack according to this example.

FIG. 29 is a sectional view taken along a-a′ of FIG. 26.

FIG. 30 is an illustration showing an example in which a cooling unit is fixed to a human body using a fixing tool.

### DESCRIPTION OF EMBODIMENTS

The definitions of terms in this application are described below. The terms shall be construed in accordance with the definitions below unless otherwise specified.

(1) A clathrate hydrate, a clathrate hydrate, a semi-clathrate hydrate, and a semi-clathrate hydrate are not distinguished in accordance with strict definitions. An aspect of the present invention is intended for a hydrate containing non-gas as a guest (guest compound).

(2) Although a heat storage material and a cold storage material are not clearly distinguished, material having a melting point of 20° C. (Celsius) or lower, which is a standard condition, is referred to as a cold storage material and material having a melting point of 20° C. (Celsius) or higher is referred to as a heat storage material in some cases.

(3) A heat storage medium or a cold storage medium is a composition of a practical form according to an aspect of the present invention and, in an aspect of the present invention, is composed of a heat (cool) storage main agent, an alkalizing agent, and a nucleating agent.

(4) A heat (cool) storage main agent refers to a composition of water and a guest compound forming a semi-clathrate hydrate (pursuant to Item (1)) containing non-gas as a guest and may be in any of a solid phase, a liquid phase, and a phase change state.

(5) The solidification temperature or the freezing temperature is the temperature at which a liquid phase transforms into a solid phase and, in an aspect of the present invention, is a value obtained in such a manner that at least 50 ml of a heat storage medium is placed in a cooling container (including a refrigerator, a freezer, and a programmable thermostatic vessel) in such a state that the heat storage medium is contained in a plastic bottle and the temperature of the heat storage medium is measured with a thermocouple while the temperature of the cooling container is being reduced. Although it is known that a supercooling phenomenon depends on the volume, experiments by the inventors have confirmed that the influence of a volume of 50 ml or more is little.

(6) The melting start temperature is the temperature determined in such a manner that the temperature at which an exothermic peak starts is extrapolated in a baseline in a DSC curve obtained by differential scanning calorimetry (DSC).

(7) A frozen state or a solidified state refers to a state in which a solid phase accounts for 95% or more of the whole volume and a slight amount of a liquid phase is separated from the solid phase, excluding a state in which solid particles are suspended or dispersed in a liquid.

(8) The latent heat capacity is a value determined from the area of an exothermic peak in a DSC curve obtained by differential scanning calorimetry (DSC). The latent heat capacity is described in the form of the heat capacity per unit volume of a heat storage medium.

(9) Positive hydration, hydrophobic hydration, or structure-making hydration is a state in which water molecules surrounding cations are strongly attracted to ions to form a highly ordered structure and therefore are more unlikely to move than bulk water molecules. Incidentally, a clathrate hydrate is hydrophobic hydration in a broad sense.

(10) Negative hydration, hydrophilic hydration, or structure-breaking hydration is a state in which water molecules surrounding cations are attracted to the cations, but not so strongly as in positive hydration, so as to be disconnected from the hydrogen bond network of bulk water molecules and therefore are more likely to move than the bulk water molecules.

(11) In general, in a heat storage tank or transport media, solid particles of a clathrate compound containing tetra-normal-butylammonium bromide as a guest are often used in a dispersed or suspended state, that is, in the form of “slurry”. In this embodiment, most of heat storage media are not in a suspended state but undergo a phase change into solids at or below the phase change temperature. This is because the heat capacity obtained in a slurry state is up to 7 cal to 11 cal 1 per g of an aqueous solution, that is, the heat capacity is very low and is insufficient for heat storage materials. In a usage pattern requiring no fluidity, a suspended state is unnecessary at a temperature not higher than the phase change temperature. A slurry state occurs when the concentration of tetra-normal-butylammonium bromide is sufficiently low, for example, 20 wt % or less. Next, embodiments of the present invention are described with reference to drawings.

[About Latent Heat Capacity and Additive]

First, the relationship between the latent heat capacity and an additive is described. Suppose that alum, which serves as a supercooling inhibitor, is added to 30 wt % TBAB. The case of adding 2% alum results in 131.84 J/g. The case of adding 3% alum results in 114.85 J/g. This shows that, in order to prevent the reduction of the latent heat capacity, it is preferable that the amount of a supercooling inhibitor is small. Next, suppose that the thickening agent CMC (F120) is added to water. The case where the additive amount is 0 results in 325 J/g. Adding 3% results in 307.4 J/g. That is, the reduction of latent heat is “−5.5%”. This shows that adding the thickening agent reduces the latent heat capacity. Thus, adding a supercooling inhibitor or an impurity generally reduces the latent heat capacity.

However, in a heat storage medium according to this embodiment, adding a supercooling inhibitor does not reduce the latent heat capacity. This is because willing separation enables the reduction of the latent heat capacity to be prevented and increases the apparent concentration of TBAB. Incidentally, in a separated state, an upper layer (first liquid layer) is a heat storage layer and a lower layer (second liquid layer) is a supercooling inhibition layer as described below.

[Configuration of Heat Storage Medium]

A heat storage medium (150) according to an aspect of the present invention is a latent heat storage medium which undergoes a phase change at a predetermined temperature and contains water, a main agent, a pH adjustor, and a nucleating agent. The main agent is a substance made of a quaternary ammonium salt and forms a semi-clathrate hydrate. Using the main agent, which forms the semi-clathrate hydrate, enables a large amount of latent heat energy to be used. The main agent is preferably tetrabutylammonium bromide (TBAB), tetrabutylammonium chloride (TBAC), tetrabutylammonium nitrate (TBAN), or tetrabutylammonium fluoride (TBAF). The congruent melting point temperature of these main agents is as described below.

TABLE 1

| | Melting | Congruent melting |
|---|---|---|
| Heat storage main agent | point | point concentration |
| Tetrabutylammonium bromide (TBAB) | 12° C. | 40.5 wt % |
| Tetrabutylammonium chloride (TBAC) | 15° C. | 34 wt % |
| Tetrabutylammonium nitrate (TBAN) | 7° C. | 39 wt % |
| Tetrabutylammonium fluoride (TBAF) | 28° C. | 33 wt % |

The pH adjustor is, for example, sodium carbonate and maintains alkalinity. The heat storage medium preferably has a pH of 10 or more. This allows a sufficiently alkaline solution to be obtained and enables cations exhibiting positive hydration to be generated. Incidentally, sodium carbonate is not a deleterious substance or a hazardous material and therefore is easier to handle as compared to sodium hydroxide.

The nucleating agent is, for example, disodium hydrogen phosphate dihydrate, disodium hydrogen phosphate heptahydrate, or disodium hydrogen phosphate dodecahydrate and generates cations exhibiting positive hydration. The above configuration allows cations, generated in an aqueous solution maintained alkaline, exhibiting positive hydration to serve as nuclei during solidification. As a result, the solidification temperature is high and the temperature difference between the solidification temperature and the melting temperature can be reduced. In addition, not only a tetragonal semi-clathrate hydrate but also an orthorhombic one can be reliably formed and can be solidified at 0° C. or higher.

The nucleating agent is preferably an anhydride or hydrate of disodium hydrogen phosphate and more preferably disodium hydrogen phosphate dodecahydrate. When both of sodium carbonate and the anhydride or hydrate of disodium hydrogen phosphate are contained in an aqueous solution, the heat storage medium can be stably solidified. Using the nucleating agent enables the effect of preventing supercooling to be enhanced.

When the main agent is tetrabutylammonium bromide (TBAB), it is preferable that the content of tetrabutylammonium bromide (TBAB) is within the range of 35 wt % to 40.5 wt %, the content of the anhydride or hydrate of disodium hydrogen phosphate is 2.5 wt % or more, and the content of sodium carbonate is 2.0 wt % or more. Alternatively, the content of tetrabutylammonium bromide (TBAB) is within the range of 30 wt % to less than 35 wt %, the content of the anhydride or hydrate of disodium hydrogen phosphate is 3.0 wt % or more, and the content of sodium carbonate is 2.0 wt % or more.

When the main agent is tetrabutylammonium chloride, it is preferable that the content of tetrabutylammonium chloride is within the range of 29 wt % to 34 wt %, the content of the anhydride or hydrate of disodium hydrogen phosphate is 2.5 wt % or more, and the content of sodium carbonate is 2.0 wt % or more. Alternatively, when the main agent is tetrabutylammonium chloride, the content of tetrabutylammonium chloride is within the range of 24 wt % to less than 29 wt %, the content of the anhydride or hydrate of disodium hydrogen phosphate is 3.0 wt % or more, and the content of sodium carbonate is 2.0 wt % or more.

When the main agent is tetrabutylammonium nitrate, it is preferable that the content of tetrabutylammonium nitrate is within the range of 34 wt % to 39 wt %, the content of the anhydride or hydrate of disodium hydrogen phosphate is 2.5 wt % or more, and the content of sodium carbonate is 2.0 wt % or more. Alternatively, when the main agent is tetrabutylammonium nitrate, the content of tetrabutylammonium nitrate is within the range of 29 wt % to less than 34 wt %, the content of the anhydride or hydrate of disodium hydrogen phosphate is 3.0 wt % or more, and the content of sodium carbonate is 2.0 wt % or more.

When the main agent is tetrabutylammonium fluoride, it is preferable that the content of tetrabutylammonium fluoride is within the range of 28 wt % to 33 wt %, the content of the anhydride or hydrate of disodium hydrogen phosphate is 2.5 wt % or more, and the content of sodium carbonate is 2.0 wt % or more. Alternatively, when the main agent is tetrabutylammonium fluoride, the content of tetrabutylammonium fluoride is within the range of 23 wt % to less than 28 wt %, the content of the anhydride or hydrate of disodium hydrogen phosphate is 3.0 wt % or more, and the content of sodium carbonate is 2.0 wt % or more.

Furthermore, the specific gravity of a second liquid layer containing the anhydride or hydrate of disodium hydrogen phosphate is higher than the specific gravity of a first liquid layer containing TBAB. This allows the first liquid layer and the second liquid layer to be in a separated state in an environment with a temperature exceeding the phase change temperature of TBAB.

Hitherto, it has been known that the supercooling inhibitor is dissolved in a liquid phase as a whole and more quickly crystallizes than the heat storage medium, which is a main agent, when the supercooling inhibitor solidifies as the temperature decreases, the crystals act as nuclei, and freezing is triggered by the nuclei. There is a difference in temperature dependence between solubilities. As the temperature decreases, the solubility decreases to serve the function of allowing a supercooling prevention agent to readily freeze.

The heat storage medium according to this embodiment separates into liquid phases because the two upper and lower layers are different in specific gravity. That is, disodium hydrogen phosphate is present as a liquid phase separated from the main agent and crystallization starts from an interface. Since the supercooling prevention agent is not contained in the main agent, the latent heat capacity does not decrease.

[Method for Producing Heat Storage Medium]

The heat storage medium can be produced in such a manner that water, the main agent (for example, TBAB, TBAC, TBAN, or TBAF), the pH adjustor (for example, sodium carbonate), and the nucleating agent (for example, disodium hydrogen phosphate dodecahydrate) are mixed together at room temperature. Upon mixing, each material is weighed such that an appropriate content is obtained, followed by mixing.

[Clathrate Hydrate]

For typical examples of the crystal structure of the clathrate hydrate, a dodecahedron, a tetradecahedron, and a hexadecahedron are known as polyhedra (cages) formed by water molecules by hydrogen bonding. Water molecules form a cavity by hydrogen bonding and hydrogen-bond to other water molecules forming a cavity to form a polyhedron. In the clathrate hydrate, crystal types called Structure I and Structure II are known.

The unit cell of each crystal type is as follows: the unit cell of Structure I is formed of 46 water molecules, six large cavities (tetradecahedra each composed of 12 five-membered rings and two six-membered rings), and two small cavities (tetradecahedra each composed of five-membered rings) and the unit cell of Structure II is formed of 136 water molecules, eight large cavities (hexadecahedra each composed of 12 five-membered rings and four six-membered rings), and 16 small cavities (tetradecahedra each composed of five-membered rings). In clathrate hydrates containing gas as a guest compound, a crystal structure formed by these unit cells is cubic as a whole.

On the other hand, when a non-gas substance which is a large molecule like the quaternary ammonium salt, which is used in an aspect of the present invention, is contained as a guest compound, the clathrate hydrate has dangling bonds because some of hydrogen bonds forming cages are broken. Semi-clathrate hydrates containing tetrabutylammonium bromide as a guest compound are classified into two types of crystal structures: one is tetragonal and the other is orthorhombic.

An orthorhombic unit cell includes cages of six dodecahedra, four tetradecahedra, and four pentadecahedra and contains two molecules of tetrabutylammonium bromide, which is a guest compound. Bromine atoms are incorporated in a cage structure and combine with water molecules. Tetrabutylammonium ions (cations) are clathrated at the centers of four cages in total: two tetradecahedra and two pentadecahedra, some of which have dangling bonds. The six dodecahedra are hollow. In a tetragonal crystal, a unit cell is formed of a combination of dodecahedra, tetradecahedra, and pentadecahedra and the dodecahedra are hollow.

Two types are described using the hydration number (molar ratio) of tetrabutylammonium bromide and water. In a tetragonal type, the average hydration number of water molecules is about 26 (a molar ratio of 1:26). In an orthorhombic type, the average hydration number thereof is about 36 (a molar ratio of 1:36). The concentration of tetrabutylammonium bromide in this case is referred to as congruent melting point concentration and is about 40 wt % and about 32 wt % in the tetragonal type and the orthorhombic type, respectively.

In this specification, a sample containing disodium hydrogen phosphate and sodium carbonate is referred to as a PC system, disodium hydrogen phosphate dodecahydrate is abbreviated as P, and sodium carbonate is abbreviated as C. The description P2.5% means that 2% disodium hydrogen phosphate dodecahydrate is added.

### Configuration of Examples and Comparative Examples

Herein, a heat storage main agent is a TBAB aqueous solution and a heat storage medium is one obtained by adding a supercooling inhibitor to the TBAB aqueous solution. A heat storage medium according to each example and the configuration of each comparative example are described below.

In Comparative Example 1, 30 g of TBAB was dissolved in 70 g of water (TBAB30 wt %).

In Comparative Example 2, 32 g of TBAB was dissolved in 68 g of water (TBAB32 wt %).

In Comparative Example 3, 35 g of TBAB was dissolved in 65 g of water (TBAB35 wt %).

In Comparative Example 4, 38 g of TBAB was dissolved in 62 g of water (TBAB38 wt %).

In Comparative Example 5, 40 g of TBAB was dissolved in 60 g of water (TBAB40 wt %).

In Comparative Example 6, 2.5 g of disodium hydrogen phosphate dodecahydrate and 2 g of sodium carbonate were added to a sample of Comparative Example 1 (TBAB30 wt %+P2.5%+C2%).

In Comparative Example 7, 2.5 g of disodium hydrogen phosphate dodecahydrate and 2 g of sodium carbonate were added to a sample of Comparative Example 2 (TBAB32 wt %+P2.5%+C2%).

In Example 1, 2.5 g of disodium hydrogen phosphate dodecahydrate and 2 g of sodium carbonate were added to a sample of Comparative Example 3 (TBAB35 wt %+P2.5%+C2%).

In Example 2, 2.5 g of disodium hydrogen phosphate dodecahydrate and 2 g of sodium carbonate were added to a sample of Comparative Example 4 (TBAB38 wt %+P2.5%+C2%).

In Example 3, 2.5 g of disodium hydrogen phosphate dodecahydrate and 2 g of sodium carbonate were added to a sample of Comparative Example 5 (TBAB40 wt %+P2.5%+C2%).

[Behavior of Each Solution]

For Examples 1-3, separation was observed at the stage of preparing a solution. In samples of Comparative Examples 1-7, no separation was observed at the stage of preparing a solution. In the case of adding a supercooling inhibitor (P2.5%+C2%), no separation occurred at 32 wt % or less and separation occurred at 35 wt % or more. In Comparative Examples 6 and 7, in which a supercooling inhibitor was added and no phase separation occurred, phase separation or crystallization was induced by freezing and melting. Phase separation is discussed in Example 7.

[Freezing-Melting Experiment]

A freezing-melting experiment was performed using a compact cool incubator (SLC-25A) manufactured by Mitsubishi Electric Engineering Co., Ltd. Upon freezing, the temperature inside was set to T=3° C. to 5° C. Upon melting, the power supply was turned off, followed by natural thawing. In comparison with general refrigerators, conditions are stringent for freezing because of no wind. Hereinafter, the temperature is represented by T.

Samples of Examples 1-2 and Comparative Examples 1-7 were frozen in a Peltier thermostatic bath. In Comparative Examples 1-5 (no supercooling inhibitor), freezing did not occur at a preset temperature of 3° C. This showed that a supercooling inhibitor was essential. In Comparative Examples 6-7 and Examples 1-3, freezing was observed at a preset temperature of 3° C. within 18 hours. After melting, in all samples of Comparative Examples 1-2 and Examples 1-3, phase separation was observed. The latent heat capacity depends on the concentration of TBAB. In the case of adding no supercooling inhibitor, the latent heat capacity peaks at TBAB 40 wt % (eutectic concentration).

[DSC Experiment]

A DSC experiment was performed using a high-sensitivity differential scanning calorimeter (Thermo plus EV02) manufactured by Rigaku Corporation. The temperature was set to change from 30° C. (5/min) to −30° C. (holding for five minutes) and to 30° C. (5/min). The latent heat capacity was calculated from the area during melting.

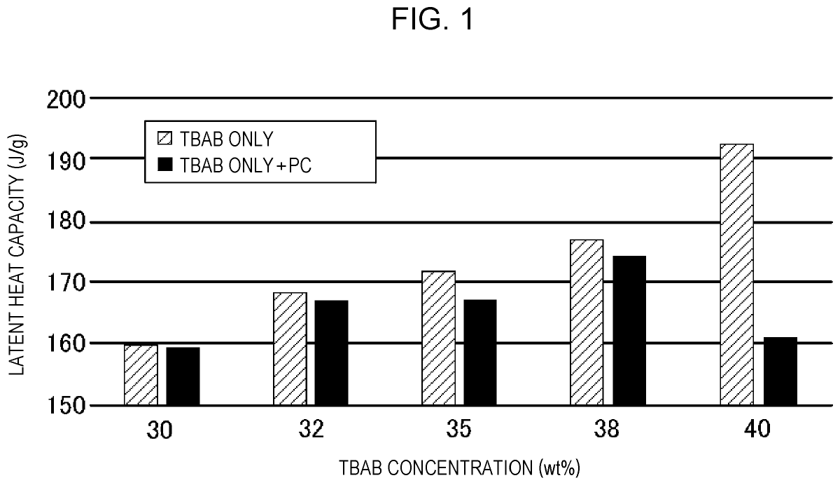

FIG. 1 is a graph showing that the latent heat capacity depends on the concentration of TBAB. As shown in FIG. 1, as the concentration of TBAB increases, the latent heat capacity due to melting at 12° C. increases and peaks at 40 wt %. The latent heat capacity decreases at a TBAB concentration of 40 wt % or more, which is not shown in the graph. On the other hand, for PC systems, the latent heat value peaks at TBAB 38 wt % and a reduction in latent heat capacity is a few percent. In this event, the amount of TBAB used is the same. However, adding (P2.5%+C2%) increases the mass of the whole. The same tendency is observed at TBAB 30 wt % to 35 wt %.

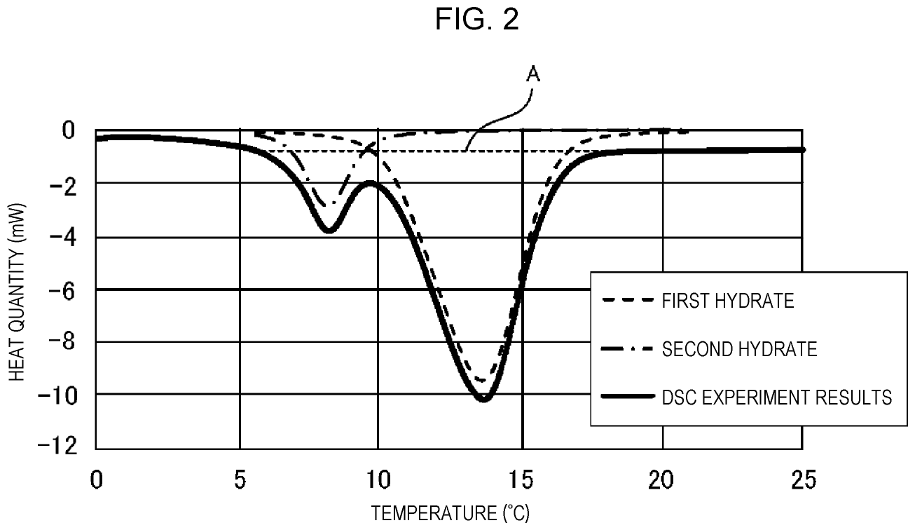

FIG. 2 is a graph showing results of DSC experiments of Examples 1-3 and Comparative Examples 1-7. As shown in FIG. 2, in the DSC experiments of Examples 1-3 and Comparative Examples 1-7, two peaks are observed. The low-temperature side originates from a second hydrate and the high-temperature side originates from a first hydrate. In this embodiment, the latent heat was defined as the area of a portion surrounded by a solid line and the finest dotted line A. The area intensity ratio between the first hydrate and the second hydrate varies depending on the concentration of TBAB.

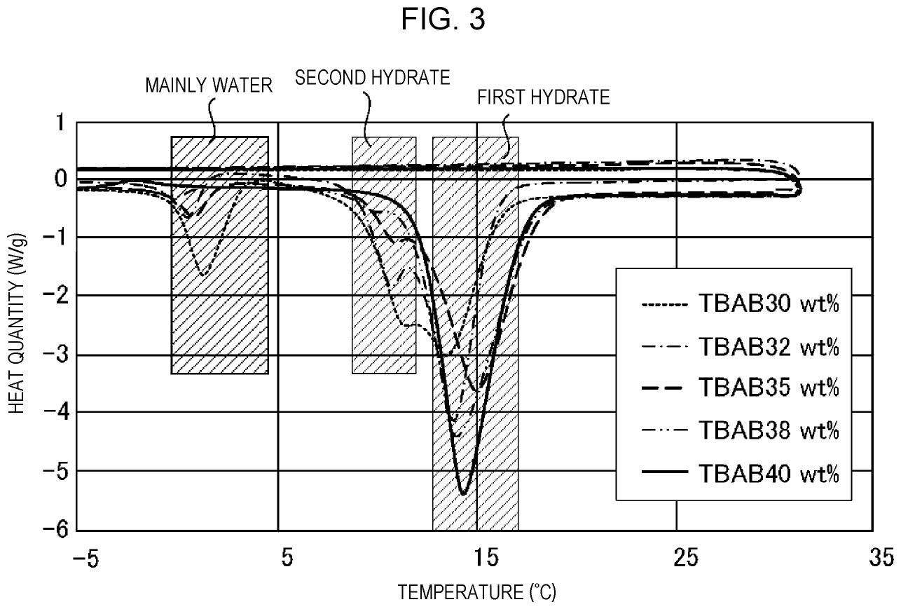

FIG. 3 is a graph showing results of DSC experiments of Comparative Examples 1-5. As shown in FIG. 3, as the concentration of TBAB increases, the latent heat capacity due to water and the latent heat capacity due to a second hydrate decrease. However, the latent heat capacity due to a first hydrate decreases. A structure originating from water or the second hydrate is not present at TBAB 40 wt %.

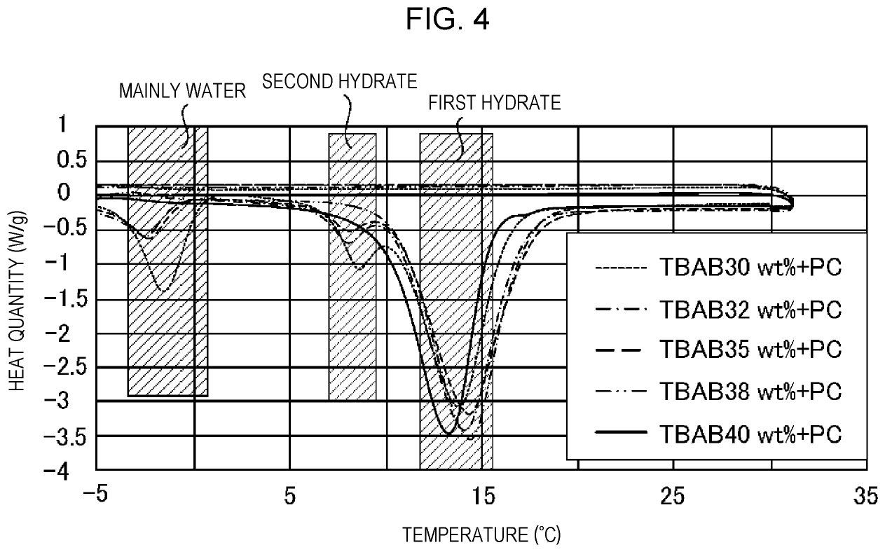

FIG. 4 is a graph showing results of DSC experiments of Examples 1-3 and Comparative Examples 6-7. As shown in FIG. 4, a structure originating from water or a second hydrate is not present in TBAB38 wt %+PC and 40 wt %+PC.

### Example 4/Comparison of Example 2 with Example 3

It is conceivable that there are limitations to the measurement of latent heat capacity in a DSC experiment because of the influence of separation. Therefore, melting behavior was observed in such a manner that samples of Examples 2 and 3 were kept cool in the same thermostatic bath preset to 3° C. and the power supply of the thermostatic bath was turned off. Incidentally, thermocouples were set such that the levels thereof were the same. Example 2 is “TBAB38 wt %+PC” and Example 3 is “TBAB40 wt %+PC”.

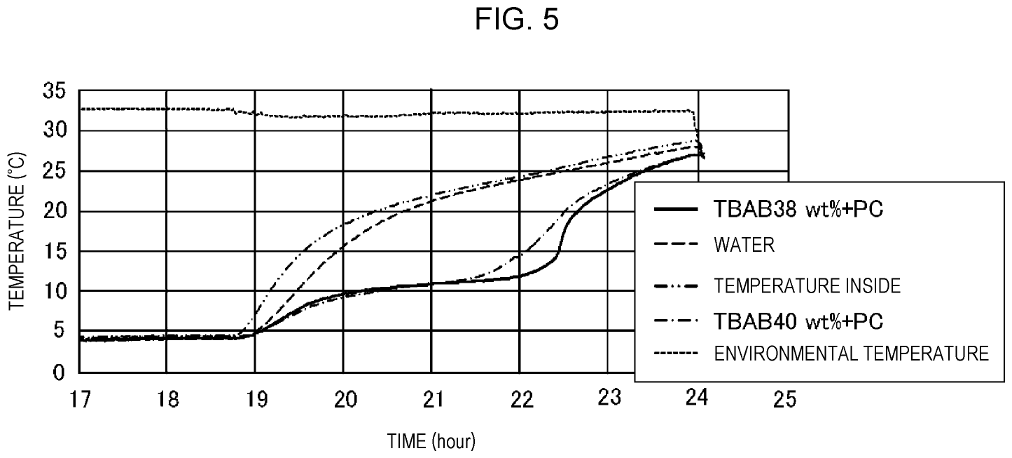

FIG. 5 is a graph showing comparative results of Examples 2 and 3. It is clear that the melting time of Example 2 (TBAB38 wt %+PC) is longer. Thus, the latent heat capacity of “TBAB38 wt %+PC” is larger. In “TBAB40 wt %+PC”, a reduction in latent heat capacity is observed, which corresponds to the fact that the amount of TBAB is large (40 wt % or more).

### Example 5/Comparison of Example 1 with Example 3

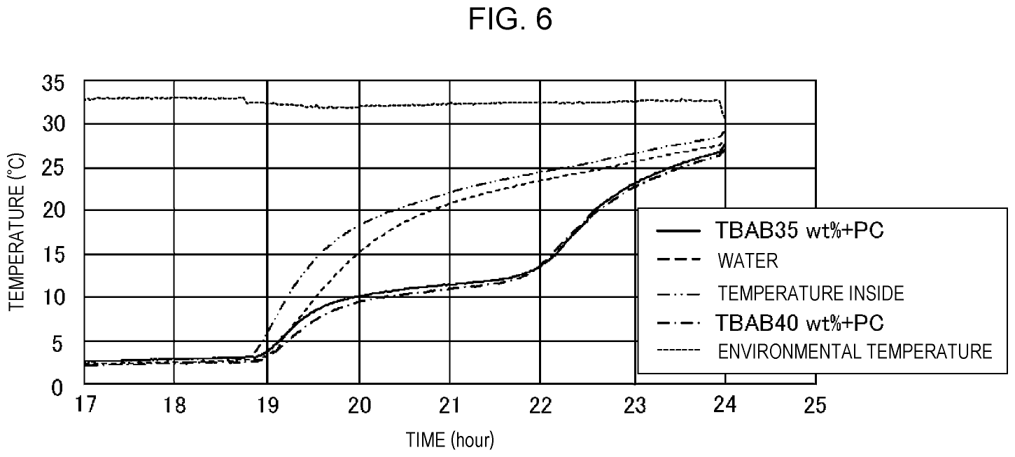

Example 1 (TBAB35 wt %+PC) and Example 3 (TBAB40 wt %+PC) were compared under the same experiment conditions as those for an experiment for comparing Examples 2 and 3. FIG. 6 is a graph showing comparative results of Examples 1 and 3. As shown in FIG. 6, according to a DSC experiment, it is clear that there is no difference in latent heat capacity between Examples 1 and 3.

### Example 6/about Freezing at 5° C.

In the comparative examples and examples described above, freezing was confirmed at a setting of 3° C. In this event, the time taken for freezing was 18 hours. Next, samples prepared in Comparative Examples 6-7 and Examples 1-3 were set in a 5° C. compact thermostatic bath. In this operation, the time taken for complete freezing was 24 hours. Although the inside temperature of refrigerators in Japan is about 3° C., the inside of refrigerators in areas in which electric power is insufficient is kept at about 5° C. in some cases. Thus, it can be said that a sample of this embodiment can be used in areas, such as Southeast Asia, unstable in electric power.

### Examples 7-9/XRD Experiment

Next, a supercooling inhibition layer which appeared in Example 1 was taken out and was subjected to an XRD experiment. In the XRD experiment, an automated horizontal multipurpose X-ray diffractometer (SmartLab) manufactured by Rigaku Corporation or an X-ray diffractometer (RINT 2500HL: low-temperature attachment) manufactured by Rigaku Corporation was used. Since a sample was liquid at room temperature and no XRD pattern could be observed, the experiment was performed at a temperature of −30° C. in a frozen state. As comparative examples, the XRD pattern of a sample (water+phosphoric acid 30%: Comparative Example 8) prepared by dissolving 30 g of disodium hydrogen phosphate dodecahydrate in 100 g of water and the XRD pattern of water (Comparative Example 9) in a frozen state are shown.

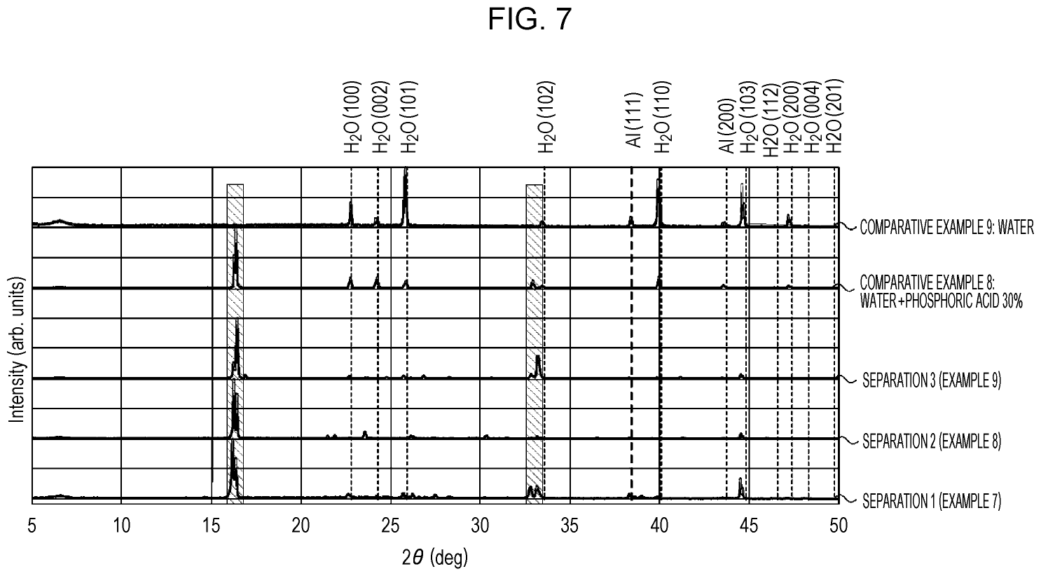

FIG. 7 is a graph showing results of the XRD experiment. The XRD patterns of Examples 7-9 do not completely coincide with each other. This is probably because a slight amount of TBAB is dissolved and the concentration of TBAB varies depending on sampling sites. What is common to the XRD patterns of Examples 7-9 is that “a structure originating from water is observed” and “a structure with high intensity is observed at 2θ=16° or 32°”. The structure observed at 2θ=16° or 32° is also observed in Comparative Example 8 (water+phosphoric acid 30%). Thus, it is conceivable that this structure originates from disodium hydrogen phosphate. However, identification could not be made in this angle range and detailed discussions were further made.

FIG. 8 is a graph showing results of XRD experiments on Examples 10 and 11. Herein, two types of solutions were prepared: one obtained by dissolving 15 g of disodium hydrogen phosphate dodecahydrate in 100 g of water (Example 10, phosphoric acid 12 water 15%) and one obtained by dissolving 6 g of disodium hydrogen phosphate dodecahydrate in 100 g of water (Example 11, phosphoric acid 12 water 15%). For Comparative Example 8 (water+phosphoric acid 30%), disodium hydrogen phosphate might possibly precipitate when the outside air temperature was low; hence, this time, experiments were performed at low concentration. The measurement temperature is −30° C. as is the case with the former.

Detailed analysis showed that structures other than water were as described below.

A structure at 2θ=16°: the (002) plane of disodium hydrogen phosphate dodecahydrate.
A structure at 2θ=32°: the (004) plane of disodium hydrogen phosphate dodecahydrate.
A structure at 2θ=50°: the (006) plane of disodium hydrogen phosphate dodecahydrate.
A structure at 2θ=68°: the (008) plane of disodium hydrogen phosphate dodecahydrate.

Thus, it became clear that, in water, a structure originating from the (002) plane of disodium hydrogen phosphate dodecahydrate appeared.

[About Behavior of Solution]

Whether a solution separated was checked. Comparative Example 10 was set to TBAB30 wt %+P2%+C2%. No separation was observed. Example 12 was set to TBAB30 wt %+P3%+C3%. Separation was observed. Example 13 was set to TBAB30 wt %+P4%+C4%. Separation was observed. This showed that the concentration causing separation corresponded to the case where 3% or more P and 3% or more C were added at TBAB30 wt % or more.

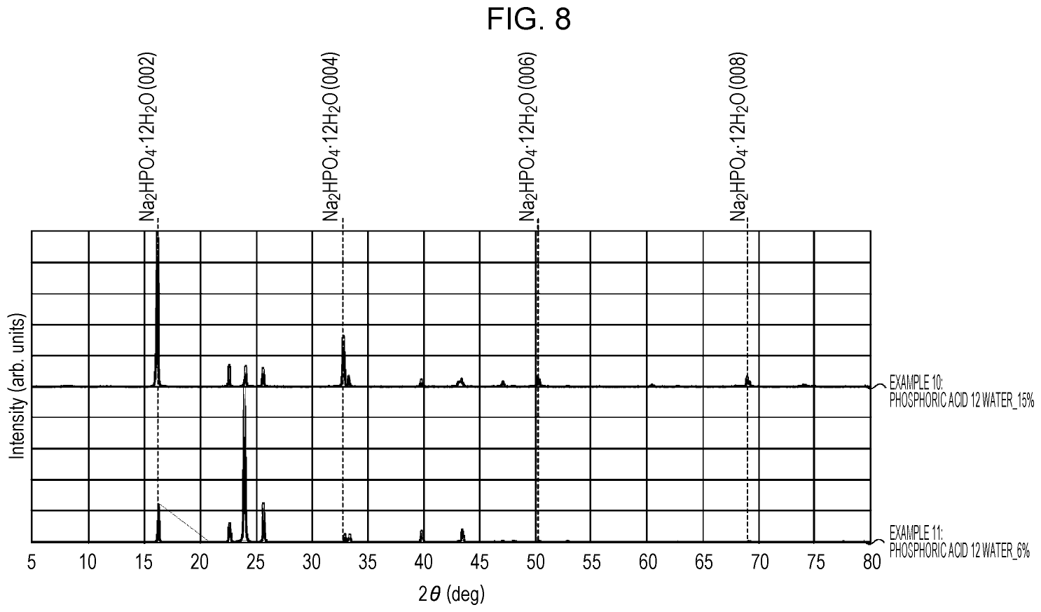

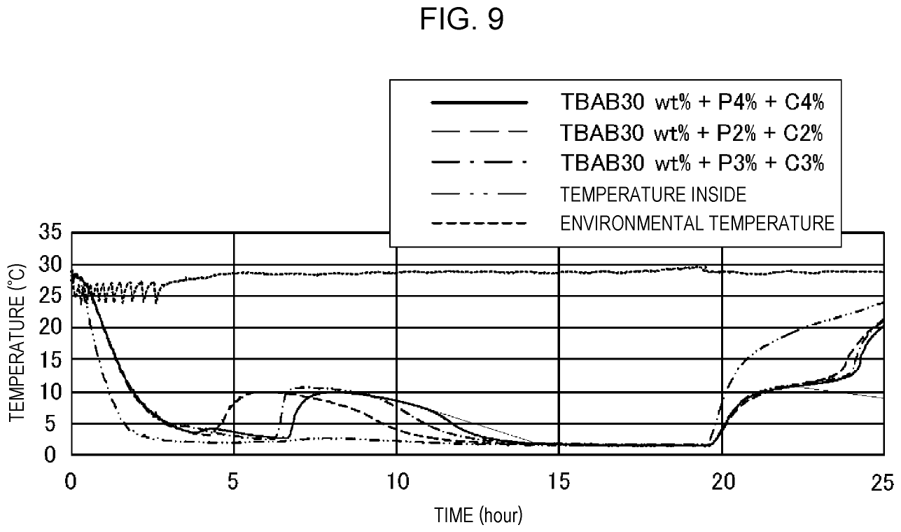

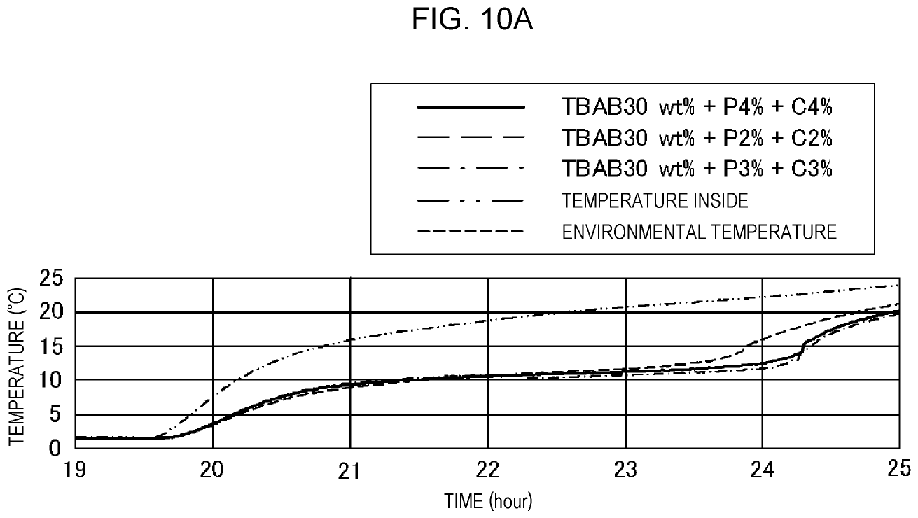

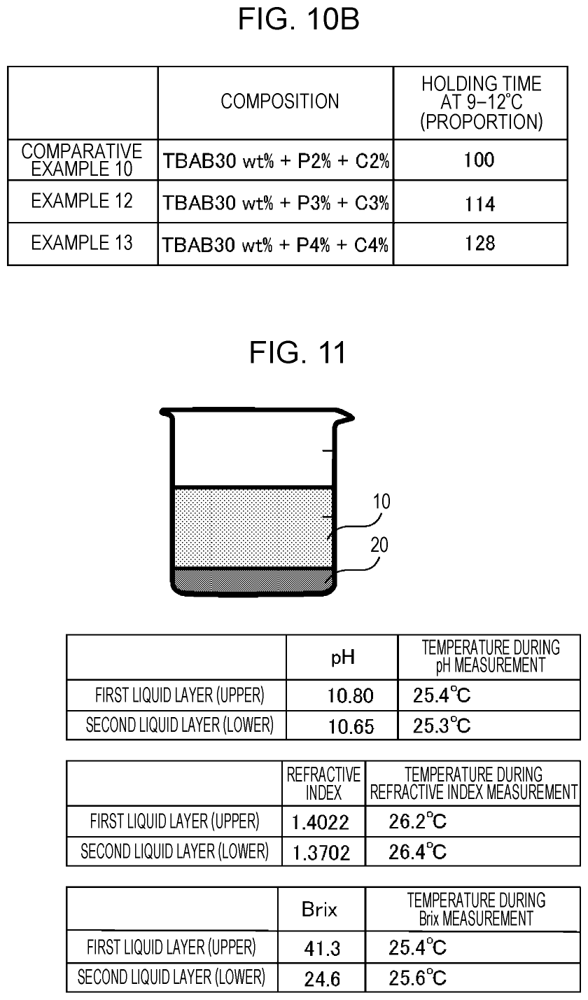

FIG. 9 is a graph showing the relationship between the time and temperature of Example 12, Example 13, and Comparative Example 10. In all of Example 12, Example 13, and Comparative Example 10, freezing was observed at 3° C. FIG. 10A is a graph showing the relationship between the time and temperature of Example 12, Example 13, and Comparative Example 10. FIG. 10B is a table showing the composition and holding time proportion of Example 12, Example 13, and Comparative Example 10. No separation occurs in Comparative Example 10 but separation occurs in Examples 12 and 13 and the holding time (=latent heat capacity) at 9-12° C. increases with the amount of separation. That is, phosphoric acid absorbs water, thereby increasing the apparent concentration of TBAB. As a result, the latent heat capacity increases.

### Example 14

FIG. 11 is an illustration showing results obtained by measuring “pH, refractive index, and Brix value” in a “TBAB38 wt %+P2.5%+C2%” system. As shown in FIG. 11, it is clear that a first liquid layer 10 which is a separated upper layer and a second liquid layer 20 which is a lower layer both exhibit alkalinity and therefore both layers contain sodium carbonate. Furthermore, it is clear that the first liquid layer 10 and the second liquid layer 20 have different refractive indices and therefore contain different solvents.

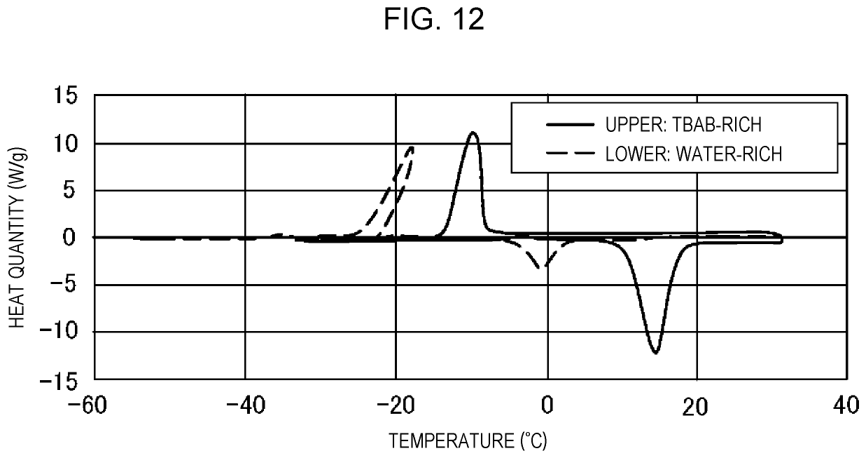

FIG. 12 shows results obtained by subjecting each of the first liquid layer 10 and the second liquid layer 20 to a DSC experiment in TBAB38 wt %+P2.5%+C2%. As shown in FIG. 12, it is clear that the first liquid layer 10 contains a large amount of TBAB and the second liquid layer 20 contains a large amount of water. Incidentally, sodium carbonate is dissolved in both layers.

[Measurement of Specific Gravity]

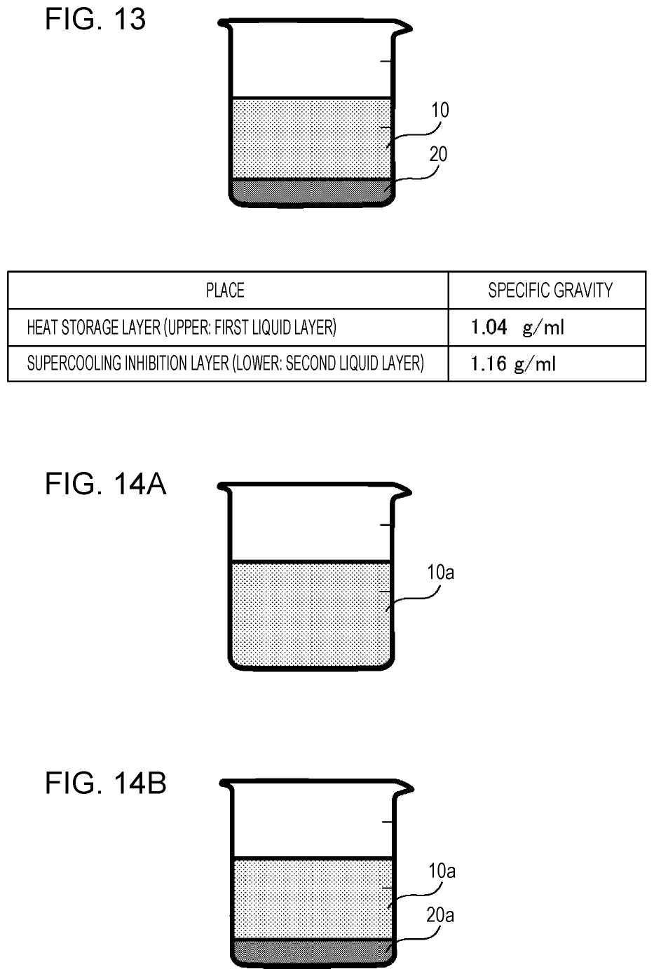

FIG. 13 is an illustration showing results obtained by measuring the specific gravity for an Example 1 (TBAB38 wt %+PC) system. It is clear that the specific gravity of a second liquid layer 20 (lower) which is a supercooling inhibition layer is higher than that of a first liquid layer 10 (upper) which is a heat storage layer. Incidentally, in an unseparated case (Comparative Example 7: TBAB32+PC), the specific gravity is 1.05 g/ml. As described above, separation occurs due to high specific gravity to provide the supercooling inhibition layer.

### Example 15

In the above description, TBAB has been used as an example for description. In this example, an aqueous solution obtained by dissolving each of TBAC, TBAN, and TBAF as a main agent in water is exemplified. As shown in FIG. 14A, in the case of a main agent only, all the aqueous solutions did not separate and were homogeneous solutions (a first liquid layer 10a). As shown in FIG. 14B, adding disodium hydrogen phosphate dodecahydrate and sodium carbonate to the aqueous solutions caused “layer separation” as was the case with an aqueous solution of TBAB (a first liquid layer 10a and a second liquid layer 20a).

### Example 16

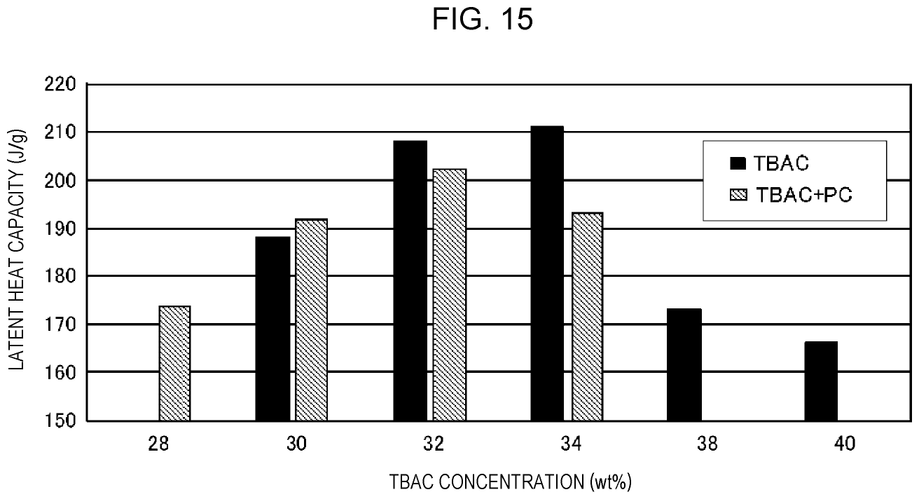

FIG. 15 is a graph showing the dependence of the latent heat capacity on the concentration of tetrabutylammonium chloride (TBAC). Herein, the case where a supercooling inhibitor is present and the case where no supercooling inhibitor is present are shown. In the case where no supercooling inhibitor is present, the latent heat capacity peaks at TBAC 34 wt %, which is the congruent melting point concentration. In this event, the latent heat capacity is 211 J/g. On the other hand, adding the supercooling inhibitor reduces the concentration at which the latent heat capacity peaks from 34 wt % to 32 wt %. As is the case with TBAB, disodium hydrogen phosphate and/or sodium carbonate hydrates in a solution or forms hydrates thereof because of a reduction in temperature to precipitate, thereby taking away water molecules used by TBAC to hydrate. As a result, water molecules are short at the congruent melting point concentration of TBAC; hence, it is conceivable that no proper semi-clathrate hydrate is formed and the latent heat capacity decreases. On the other hand, water taken away by disodium hydrogen phosphate and/or sodium carbonate is supplemented by adjusting the concentration below the congruent melting point concentration; hence, a proper semi-clathrate hydrate is formed and the latent heat capacity peaks. In this event, the latent heat capacity is 202 J/g and the rate of decrease with respect to the maximum in the case where no supercooling inhibitor is present is about 4%. Therefore, it can be said that, even if the supercooling inhibitor is added, the latent heat capacity does not significantly decrease.

### Example 17

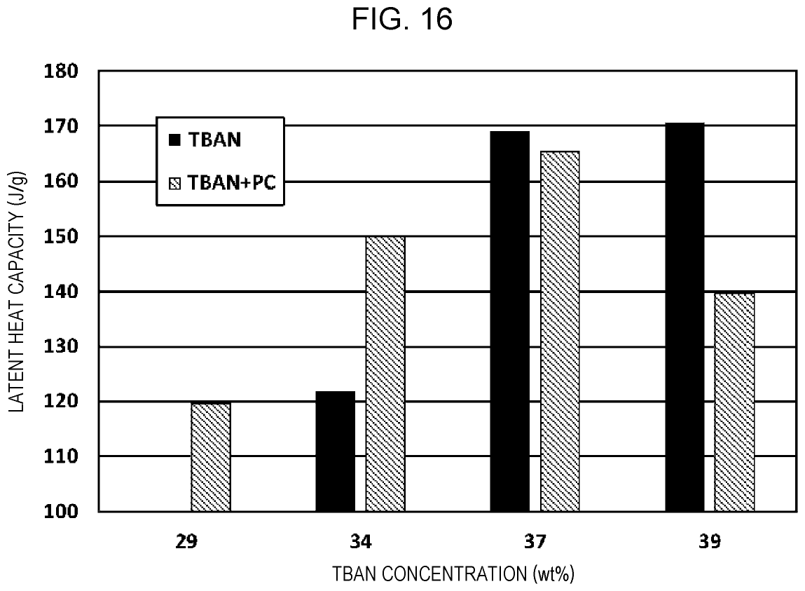

FIG. 16 is a graph showing the dependence of the latent heat capacity on the concentration of tetrabutylammonium nitrate (TBAN). Herein, the case where a supercooling inhibitor is present and the case where no supercooling inhibitor is present are shown. In the case where no supercooling inhibitor is present, the latent heat capacity peaks at TBAN 39 wt %, which is the congruent melting point concentration. In this event, the latent heat capacity is 170 J/g. On the other hand, adding the supercooling inhibitor reduces the concentration at which the latent heat capacity peaks from 39 wt % to 37 wt %. As is the case with TBAB, disodium hydrogen phosphate and/or sodium carbonate hydrates in a solution or forms hydrates thereof because of a reduction in temperature to precipitate, thereby taking away water molecules used by TBAN to hydrate. As a result, water molecules are short at the congruent melting point concentration of TBAN; hence, it is conceivable that no proper semi-clathrate hydrate is formed and the latent heat capacity decreases. On the other hand, water taken away by disodium hydrogen phosphate and/or sodium carbonate is supplemented by adjusting the concentration below the congruent melting point concentration; hence, a proper semi-clathrate hydrate is formed and the latent heat capacity peaks. In this event, the latent heat capacity is 165 J/g and the rate of decrease with respect to the maximum in the case where no supercooling inhibitor is present is about 3%. Therefore, it can be said that, even if the supercooling inhibitor is added, the latent heat capacity does not significantly decrease.

### Example 18

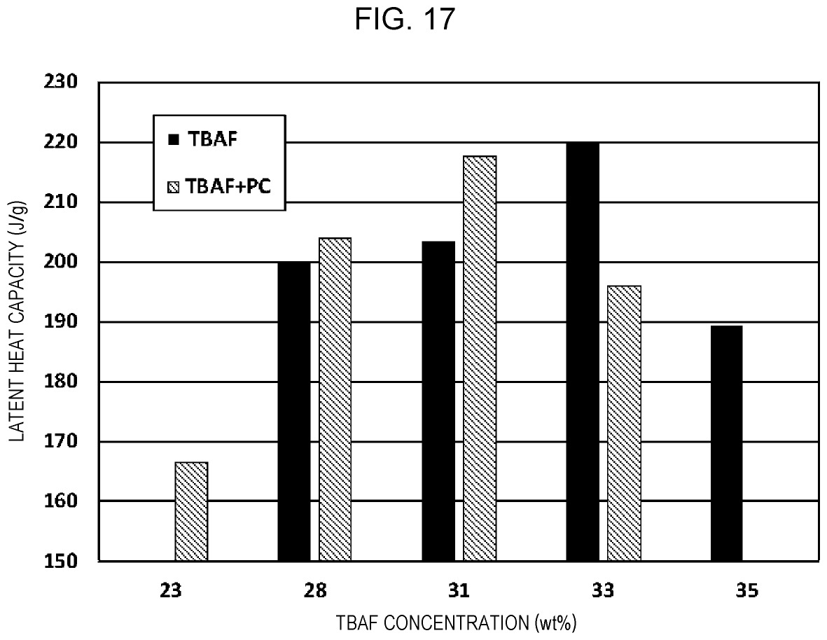

FIG. 17 is a graph showing the dependence of the latent heat capacity on the concentration of tetrabutylammonium fluoride (TBAF). Herein, the case where a supercooling inhibitor is present and the case where no supercooling inhibitor is present are shown. In the case where no supercooling inhibitor is present, the latent heat capacity peaks at TBAF 33 wt %, which is the congruent melting point concentration. In this event, the latent heat capacity is 220 J/g. On the other hand, adding the supercooling inhibitor reduces the concentration at which the latent heat capacity peaks from 33 wt % to 31 wt %. As is the case with TBAB, disodium hydrogen phosphate and/or sodium carbonate hydrates in a solution or forms hydrates thereof because of a reduction in temperature to precipitate, thereby taking away water molecules used by TBAF to hydrate. As a result, water molecules are short at the congruent melting point concentration of TBAF; hence, it is conceivable that no proper semi-clathrate hydrate is formed and the latent heat capacity decreases. On the other hand, water taken away by disodium hydrogen phosphate and/or sodium carbonate is supplemented by adjusting the concentration below the congruent melting point concentration; hence, a proper semi-clathrate hydrate is formed and the latent heat capacity peaks. In this event, the latent heat capacity is 217 J/g and the rate of decrease with respect to the maximum in the case where no supercooling inhibitor is present is about 2%. Therefore, it can be said that, even if the supercooling inhibitor is added, the latent heat capacity does not significantly decrease.

### Example 19

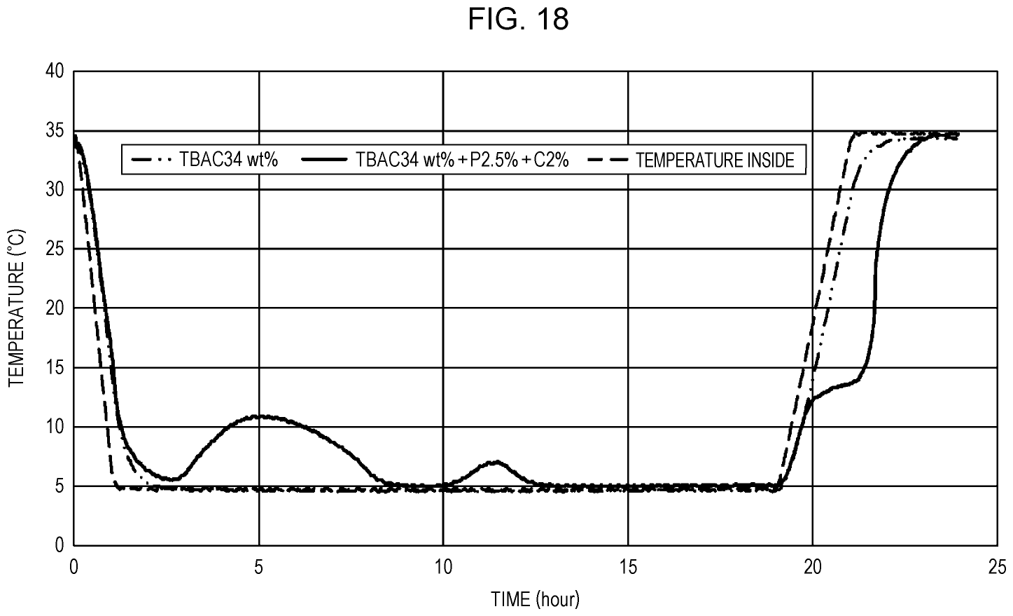

FIG. 18 is a graph showing results of an experiment for measuring the change in temperature of aqueous solutions of TBAC. Herein, a 34 wt % aqueous solution of TBAC (TBAC34 wt %) was prepared and “TBAC34 wt %+P2.5%+C2%” was prepared by adding 2.5% disodium hydrogen phosphate dodecahydrate and 2% sodium carbonate to the solution. Changes in temperature were measured by sequentially varying the temperature in a compact thermostatic bath to 35° C., 5° C., and 35° C. In the case where no supercooling inhibitor was present (TBAC34 wt %), the TBAC aqueous solution did not freeze. On the other hand, in the case where a supercooling inhibitor was present (TBAC32 wt %+P2.5%+C2%), an exothermic peak was observed at “T=5° C.”, whereby freezing was confirmed. In the increase of temperature, one containing the supercooling inhibitor exhibited melting behavior (phase transition) at “T=14° C.”. On the other hand, the TBAC aqueous solution did not freeze under these conditions and therefore the phenomenon was not seen.

From the above, it became clear that a combination of disodium hydrogen phosphate dodecahydrate and sodium carbonate had the effect of preventing supercooling on the TBAC aqueous solution. This allows the effect of reducing power consumption for cooling to be expected. In the case where no supercooling inhibitor is present, the freezing temperature of the TBAC aqueous solution is about −3° C. In this case, a negative temperature is necessary for freezing and therefore power consumption is further necessary.

### Example 20

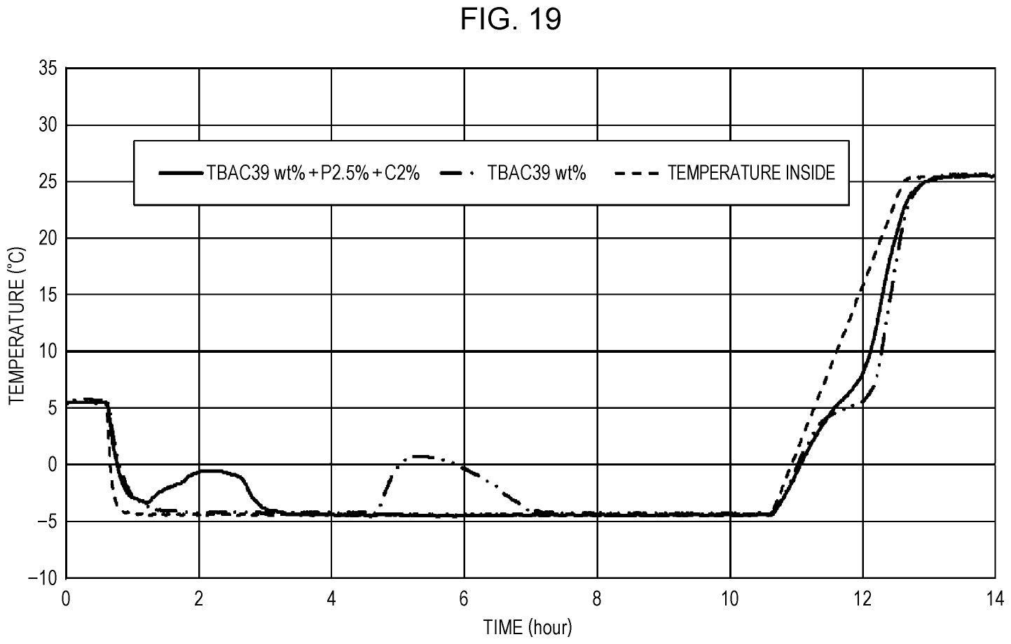

FIG. 19 is a graph showing results of an experiment for measuring the change in temperature of aqueous solutions of TBAN. Herein, a 39 wt % aqueous solution of TBAN (TBAN39 wt %) was prepared and “TBAN39 wt %+P2.5%+C2%” was prepared by adding 2.5% disodium hydrogen phosphate dodecahydrate and 2% sodium carbonate to the solution. Changes in temperature were measured by sequentially varying the temperature in a compact thermostatic bath to 5° C., −5° C., and 25° C. In the case where a supercooling inhibitor was present (TBAN39 wt %+P2.5%+C2%), freezing primarily occurred at T=−3° C. One containing no supercooling inhibitor froze at T=−5° C. That is, it was confirmed that adding the supercooling inhibitor allowed short freezing time and high freezing temperature to be achieved. On the other hand, in the increase of temperature, the TBAN aqueous solution exhibited melting behavior (phase transition) at T=4° C. and one containing the supercooling inhibitor exhibited melting behavior accompanied by a gradual increase in temperature at T=4-7° C. This corresponds to the results shown in FIG. 17. The TBAN aqueous solution used in this example has congruent melting point concentration and disodium hydrogen phosphate and/or sodium carbonate takes away water, thereby causing the increase in the apparent concentration of TBAN.

From the above, it became clear that a combination of disodium hydrogen phosphate dodecahydrate and sodium carbonate had the effect of preventing supercooling on the TBAN aqueous solution. This allows the effect of reducing power consumption for cooling to be expected. In the case where no supercooling inhibitor is present, the freezing temperature of the TBAN aqueous solution is about −10° C. In this case, a negative temperature is necessary for freezing and therefore power consumption is further necessary.

### Example 21

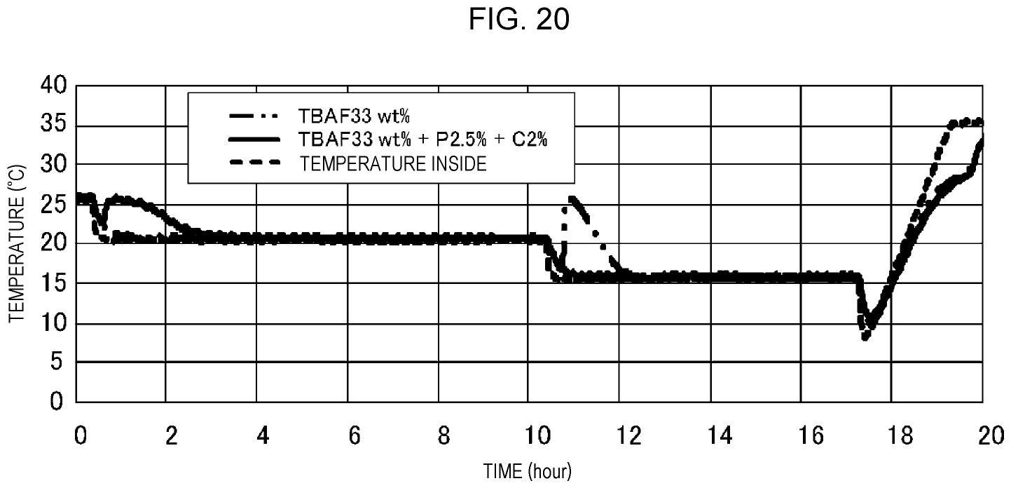

FIG. 20 is a graph showing results of an experiment for measuring the change in temperature of aqueous solutions of TBAF. Herein, a 33 wt % aqueous solution of TBAF (TBAF33 wt %) was prepared and “TBAF33 wt %+P2.5%+C2%” was prepared by adding 2.5% disodium hydrogen phosphate dodecahydrate and 2% sodium carbonate to the solution. Changes in temperature were measured by sequentially varying the temperature in a compact thermostatic bath to 25° C., 15° C., and 35° C. In the case where no supercooling inhibitor was present (TBAN33 wt %), TBAF froze at “T=16° C.”. On the other hand, in the case where a supercooling inhibitor was present (TBAF33 wt %+P2.5%+C2%), freezing occurred at “T=22° C.”. That is, freezing occurred at a temperature 6° C. higher than that in the case where no supercooling inhibitor was present.

From the above, it became clear that a combination of disodium hydrogen phosphate dodecahydrate and sodium carbonate had the effect of preventing supercooling on the TBAF aqueous solution.

### Example 22

Next, results obtained by checking the separation of TBAC are described. First, (a) 2.5% disodium hydrogen phosphate dodecahydrate and 2% sodium carbonate were added to TBAC34 wt %. This solution underwent phase separation. An upper layer originates mainly from an aqueous solution of TBAC and a lower layer originates mainly from disodium hydrogen phosphate. Next, (b) 2.5% disodium hydrogen phosphate dodecahydrate and 2% sodium carbonate were added to TBAC28 wt %. This solution underwent phase separation. An upper layer originates mainly from an aqueous solution of TBAC and a lower layer originates mainly from disodium hydrogen phosphate. Next, (c) 3% disodium hydrogen phosphate dodecahydrate and 4% sodium carbonate were added to TBAC24 wt %. This solution underwent phase separation. An upper layer originates mainly from an aqueous solution of TBAC and a lower layer originates mainly from disodium hydrogen phosphate. As described above, in all Models (a) to (c), the separation of TBAC was observed. In all Models (a) to (c), a freezing experiment was attempted in substantially the same manner as that used in Example 19, whereby it was confirmed that the freezing temperature was higher as compared to that of one containing no supercooling inhibitor.

### Example 23

Next, results obtained by checking the separation of TBAN are described. First, (a) 2.5% disodium hydrogen phosphate dodecahydrate and 2% sodium carbonate were added to TBAN39 wt %. This solution underwent phase separation. An upper layer originates mainly from an aqueous solution of TBAN and a lower layer originates mainly from disodium hydrogen phosphate. Next, (b) 2.5% disodium hydrogen phosphate dodecahydrate and 2% sodium carbonate were added to TBAN34 wt %. This solution underwent phase separation. An upper layer originates mainly from an aqueous solution of TBAN and a lower layer originates mainly from disodium hydrogen phosphate. Next, (c) 3.0% disodium hydrogen phosphate dodecahydrate and 2.5% sodium carbonate were added to TBAN29 wt %. This solution underwent phase separation. An upper layer originates mainly from an aqueous solution of TBAN and a lower layer originates mainly from disodium hydrogen phosphate. As described above, in all Models (a) to (c), the separation of TBAN was observed. In all Models (a) to (c), a freezing experiment was attempted in substantially the same manner as that used in Example 20, whereby it was confirmed that the freezing temperature was higher as compared to that of one containing no supercooling inhibitor.

### Example 24

Next, results obtained by checking the separation of TBAF are described. First, (a) 2.5% disodium hydrogen phosphate dodecahydrate and 2% sodium carbonate were added to TBAF33 wt %. This solution underwent phase separation. An upper layer originates mainly from an aqueous solution of TBAF and a lower layer originates mainly from disodium hydrogen phosphate. Next, (b) 3.0% disodium hydrogen phosphate dodecahydrate and 2% sodium carbonate were added to TBAF23 wt %. This solution underwent phase separation. An upper layer originates mainly from an aqueous solution of TBAF and a lower layer originates mainly from disodium hydrogen phosphate. As described above, in all Models (a) and (b), the separation of TBAF was observed. In all Models (a) to (c), a freezing experiment was attempted in substantially the same manner as that used in Example 21, whereby it was confirmed that the freezing temperature was higher as compared to that of one containing no supercooling inhibitor.

### Example 25

[Configuration of Cooling Pack]

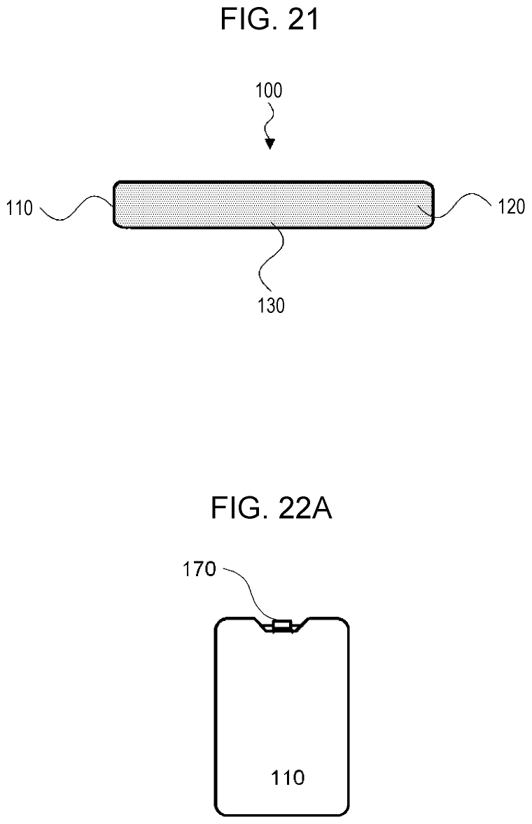

FIG. 21 is a sectional view of a cooling pack 100 according to this example. As shown in FIG. 21, the cooling pack 100 according to this example includes a housing section 120 which is a hollow-structured region in a cooling pack body 110 and also includes a heat storage layer 130 in the housing section 120.

The cooling pack body 110 includes the housing section 120, which has a hollow structure for containing the heat storage layer 130. The cooling pack body 110 can be formed from a resin material such as polyethylene, polypropylene, polyester, polyurethane, polycarbonate, polyvinyl chloride, or polyamide; metal such as aluminium, stainless steel, copper, or silver; or an inorganic material such as glass, porcelain, or ceramic. From the viewpoint of the ease of preparing the hollow structure and durability, the resin material is preferable. The cooling pack body 110 may be wrapped in a film of polyethylene, polypropylene, polyester, polyurethane, polycarbonate, polyvinyl chloride, polyamide, or the like. The film is preferably provided with a thin film of aluminium or silicon dioxide for the purpose of enhancing the durability and barrier properties of the film. Furthermore, a seal made of a heat-sensitive material sensitive to temperature is preferably attached to the cooling pack body 110 because the temperature of the cooling pack can be judged.

The heat storage layer 130 contains a heat storage medium 150 according to this embodiment. Material for forming the heat storage layer 130 preferably contains a preservative or an antibacterial agent. The material for forming the heat storage layer 130 may contain a thickening agent such as xanthan gum, guar gum, carboxymethylcellulose, or sodium polyacrylate. Material of the present invention is not limited to the above-exemplified material.

Bringing the cooling pack of the present invention close to or into contact with an article enables the temperature of the article to be adjusted or enables the article to be cooled in the vicinity of the melting point of the heat storage medium according to the present invention.

[Method for Manufacturing Cooling Pack]

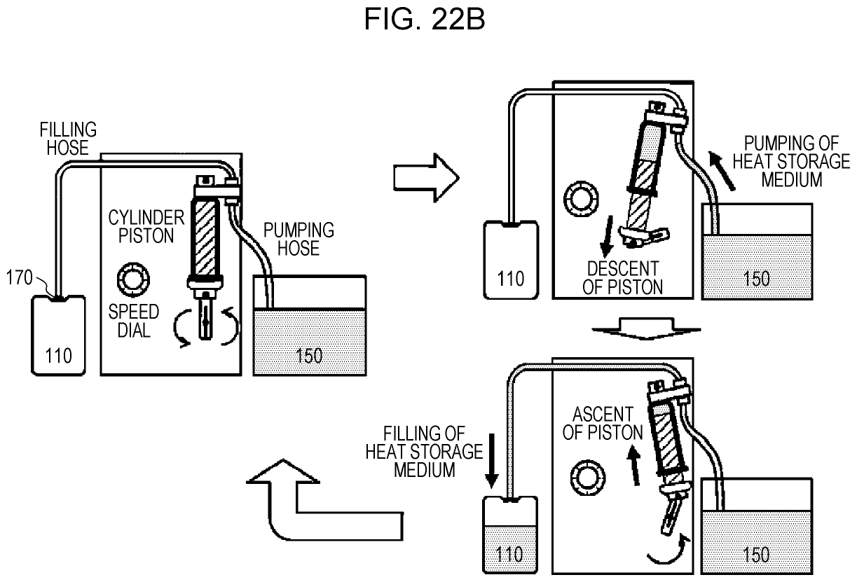

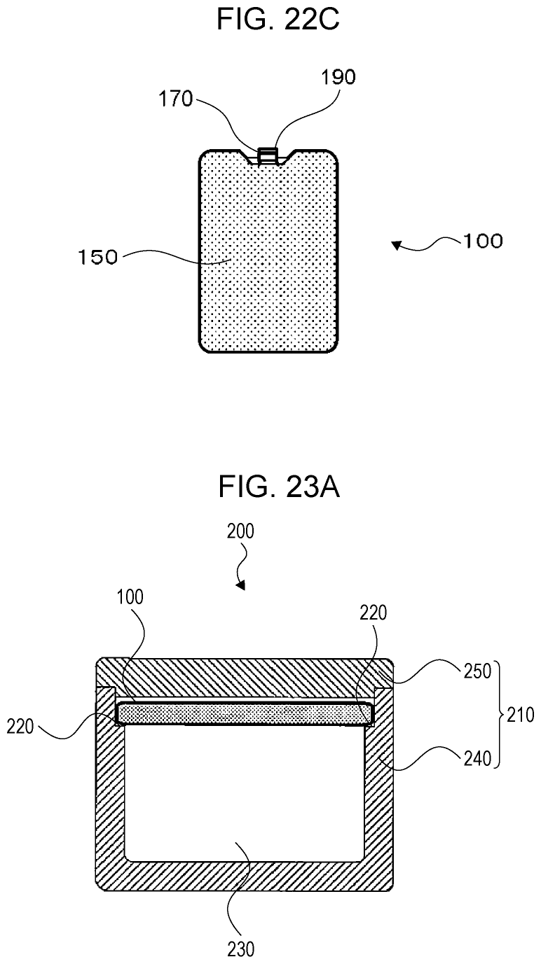

Next, a method for manufacturing the cooling pack 100 according to this example is described. FIGS. 22A to 22C are conceptual views showing steps for manufacturing the cooling pack 100 according to this example. First, as shown in FIG. 22A, the cooling pack body 110 is prepared so as to have a region with a hollow structure. The cooling pack body 110 is preferably provided with an inlet 170 through which the heat storage medium 150 can be injected. Next, the heat storage medium 150 is injected. Although an injection method is no object, an injection method in which a cylinder pump or a mohno pump is used is preferable. FIG. 20B shows an example in which the cylinder pump is used. As shown in FIG. 22B, a filling hose of the cylinder pump is set in the inlet 170 of the cooling pack body 110 and a pumping hose is set in a container containing the heat storage medium 150. Next, after the heat storage medium 150 is pumped by causing a piston of the cylinder pump to descend such that the heat storage medium 150 is filled in the piston, the heat storage medium 150 is injected into the cooling pack body 110 by causing the piston to ascend.

As shown in FIG. 22C, a plug 190 is fit into the inlet 170 of the cooling pack body 110. Examples of a method for fitting the plug 190 include a method for fitting an airtight plug by an existing technique such as ultrasonic welding or heat welding and a method in which a screw plug can be freely loosened or tightened with hand. Fitting an airtight plug by ultrasonic welding or heat welding is preferable because the heat storage medium 150 or the like will not possibly leak.

Finally, the cooling pack 100 is left stationary in an environment with a temperature not higher than the solidification temperature of the heat storage medium 150, whereby the heat storage medium 150 is solidified. Through these steps, the cooling pack 100 according to this example is manufactured. As described herein, the heat storage medium 150 may be solidified before the cooling pack 100 is supported on a logistics package 200 below. In the case where the logistics package 200 can be kept in an environment with a temperature not higher than the solidification temperature of the heat storage medium 150 in an initial stage of a logistics process, the heat storage medium 150 in the cooling pack 100 may be solidified in this stage. Incidentally, the technical scope of the present invention is not limited to the above embodiment and various modifications can be made without departing from the spirit of the present invention.

### Example 26

[Composition of Logistics Package]

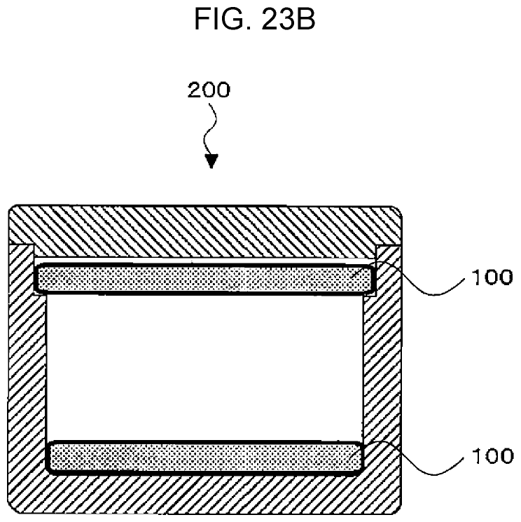

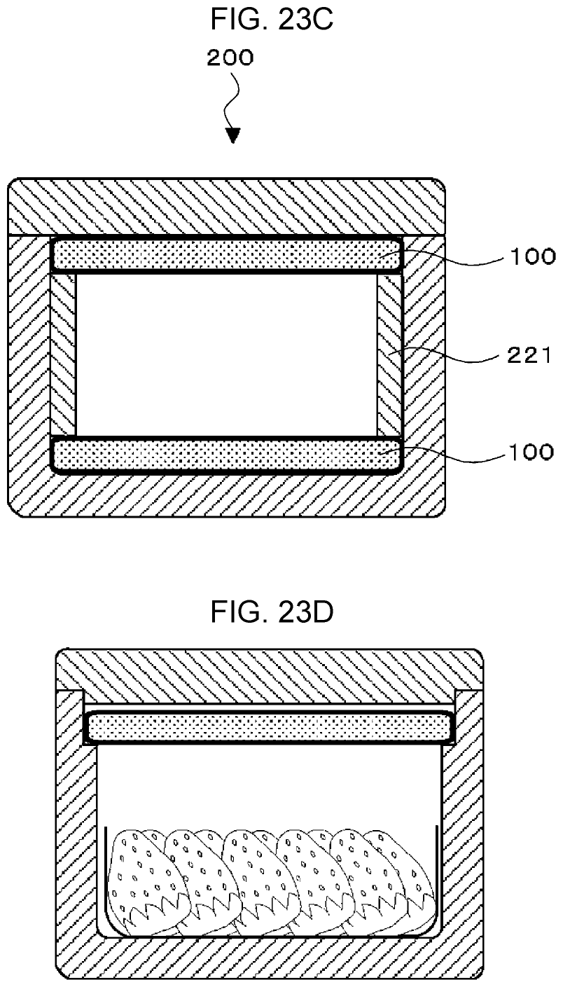

FIG. 23A is a sectional view of a logistics package 200 according to this example. The logistics package 200 includes a logistics package body 210, a cooling pack-holding section 220 which is placed in the logistics package body 210 and which holds a cooling pack, a cooling pack 100, and an article-housing section 230 which is placed in the logistics package body 210 and which houses an article (cooling object).

The logistics package body 210 is composed of a housing section 240 and a lid section 250. The housing section 240 has an opening portion for loading and unloading the article and the cooling pack 100. The lid section 250 blocks the opening portion. The housing section 240 and the lid section 250 may be connected to or separated from each other. In order to reduce the passage of heat from the inside of the logistics package 200, the lid section 250 preferably has a structure in close contact with the housing section 240.

The logistics package body 210 is preferably formed of a heat-insulating material such as foamed polystyrene, urethane foam, or a vacuum insulation material. A heat-insulating layer formed of the heat-insulating material may be placed inside or outside a body formed of material taking no account of heat-insulating properties. The logistics package body 210 may have a size capable of being carried by a person. For example, a huge vessel such as a container may have a function as the logistics package body 210. The logistics package may be a container, such as a reefer container, equipped with a cooling system.

The cooling pack-holding section 220 is placed in the logistics package body 210. The logistics package 200 is used in such a manner that the cooling pack 100 is supported on the cooling pack-holding section 220. This allows the inside of the logistics package body 210 to be maintained close to the melting point of the heat storage medium 150 of the cooling pack 100. The cooling pack-holding section 220 may have a structure to which the cooling pack 100 can be fixed. The cooling pack 100 may be placed in the logistics package body 210 or may serve as the logistics package 200.

The article-housing section 230 is placed in the logistics package body 210 and houses an article that should be maintained in a temperature range covering the melting point of the heat storage medium 150. This allows the article to be maintained close to the melting point of the heat storage medium 150. FIGS. 21B and 23C are sectional views of variations of the logistics package 200 according to this example. As shown in FIGS. 23B and 23C, a plurality of cooling packs 100 may be arranged. As shown in FIG. 23C, the cooling packs 100 may be supported with a cooling pack-holding member 221. FIG. 23D is a conceptual view showing the usage state of the cooling pack 100 and logistics package 200 according to this example. As shown in FIG. 23D, the cooling pack 100 and logistics package 200 according to this example are used in such a state that articles and the cooling pack 100 are packed in the logistics package 200.

### Example 27

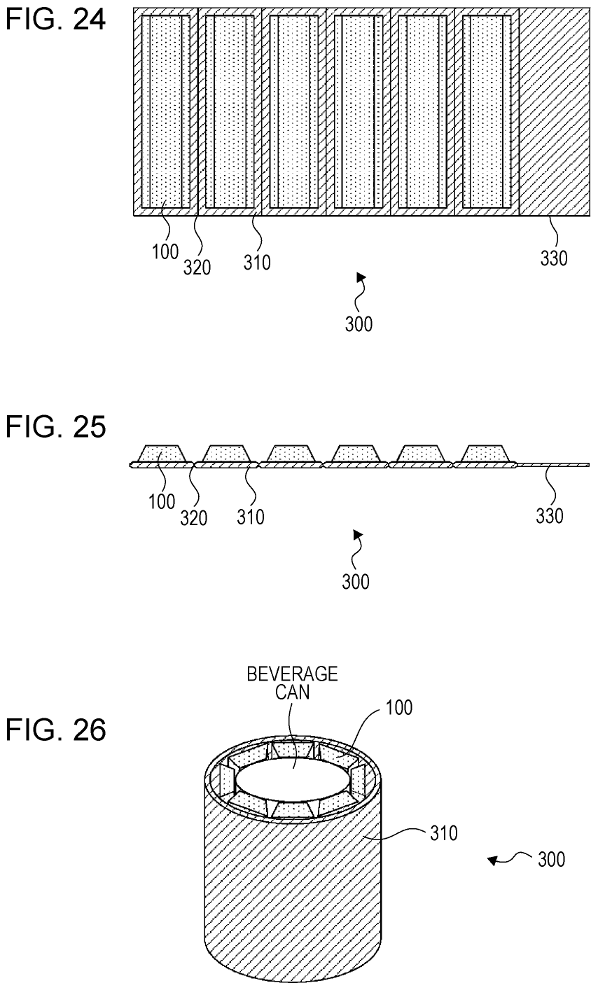

This example relates to a cooling unit including a plurality of cooling packs containing the heat storage medium according to this embodiment. FIGS. 24 and 25 are schematic views each showing an example of the cooling unit 300 according to this example. The cooling unit 300 according to this embodiment includes a plurality of cooling packs 100 according to Example 23 and cooling pack supports 310.

The cooling packs 100 are strip-shaped. The cooling packs 100 are trapezoid-shaped in cross section as shown in FIGS. 24 and 25 and may have another shape. When a cooling object is, for example, a cylindrical can or the like, a contact surface thereof may be curved for the purpose of increasing the contact area of the cooling object. The longitudinal thickness thereof may be varied so as to fit to a wine bottle or the like. FIGS. 24 and 25 each show an example in which six of the cooling packs 100 are used. The cooling packs 100 may be used as many as necessary depending on the cooling object, which is cooled by the cooling unit 300.

Each of the cooling pack supports 310 is disposed along the periphery of a corresponding one of the cooling packs 100. The cooling pack supports 310 support the cooling packs 100 and bring the cooling packs 100 close to or into contact with the cooling object. The cooling pack supports 310 may be detachably attached to the cooling packs 100 or may be fixed to the cooling packs 100 so as to be united therewith. When the cooling packs 100 are detachable, the number of the cooling packs 100 used can be varied depending on the length of a portion of the cooling object at which the cooling unit 300 is disposed. The cooling packs 100 can be solidified in an environment with a temperature not higher than the solidification temperature.

The cooling pack supports 310 are preferably formed of one, such as foamed polystyrene, urethane foam, or glass wool, having heat-insulating properties, the one preventing heat exchange with outside air. A surface may be formed of material taking no account of heat-insulating properties and another surface may be formed of material having heat-insulating properties.

The cooling pack supports 310 preferably include joint mechanisms 320 connecting the neighboring cooling packs 100. This allows the cooling packs 100 to be united with each other and also allows the cooling packs 100 to have the degree of freedom; hence, operability upon disposing the cooling packs 100 at the cooling object is enhanced. FIGS. 22 and 23 show a configuration in which the cooling pack supports 310 are formed of a plurality of plate-shaped materials and portions connecting the plate-shaped materials are equipped with the joint mechanisms 320. When the cooling pack supports 310 are formed of a flexible material, a configuration in which the joint mechanisms 320 are due to the flexibility of the material itself may be used.

The cooling pack supports 310 are sheet-shaped and can be wound around the cooling object when the cooling unit 300 is disposed at the cooling object. In this case, a fixing mechanism 330 is preferably placed such that the fixing mechanism 330 can be fixed at an arbitrary position depending on the length of a portion of the cooling object at which the cooling unit 300 is disposed. The fixing mechanism 330 used may be, for example, a hook-and-loop fastener. In the case of using the hook-and-loop fastener, at least one end portion of each cooling pack support 310 is preferably formed of a flexible material.

The cooling pack supports 310 are formed into a cylinder and can be configured such that the cooling object is put in the cavity of the cylinder of the cooling unit 300 when the cooling unit 300 is disposed at the cooling object. In this case, each cooling pack support 310 preferably includes at least one portion formed of an elastic material for the purpose of allowing the size of the cooling object to have a certain range. This enables the cooling pack 100 to be brought into contact with the cooling object, which has a size in a certain range, with elastic force. Such a configuration can be obtained by forming the joint mechanisms 320 from, for example, rubber.

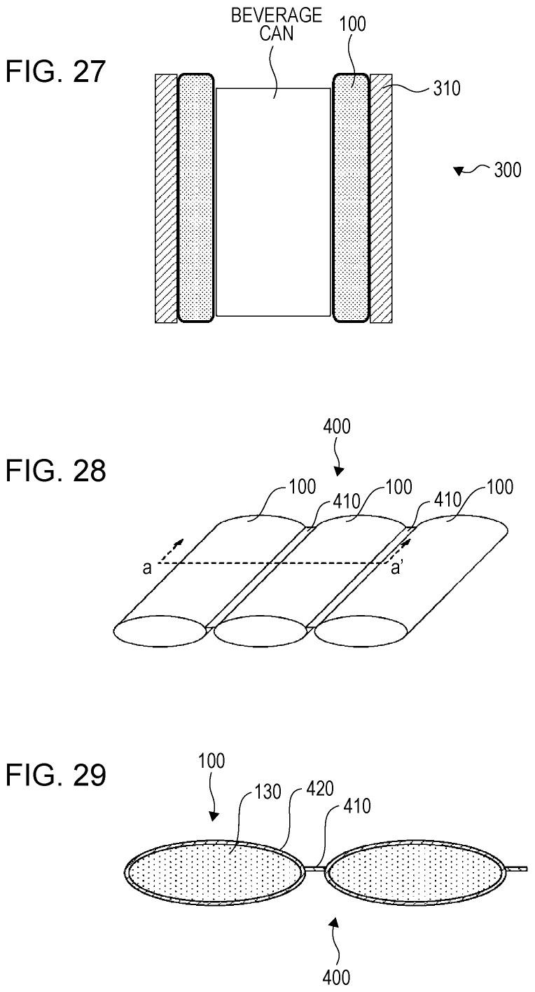

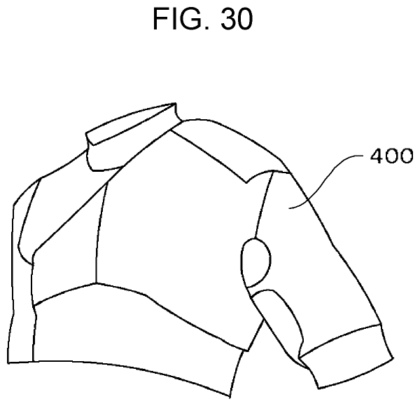

FIG. 26 is a conceptual view showing an example of the usage state of the cooling unit 300 according to this example. FIG. 27 is a sectional view showing an example of the usage state of the cooling unit 300 according to this example. As shown in FIGS. 26 and 27, the cooling unit 300 is disposed around the cooling object, whereby the cooling packs 100 are brought close to or into contact with the cooling object. As a result, the cooling object can be maintained close to the melting point of the cooling packs 100.

### Example 28

Example 28 relates to a cooling unit including a plurality of cooling packs containing the heat storage medium according to this embodiment. FIGS. 28 and 29 are schematic views each showing an example of a cooling unit 400 according to this example. The cooling unit 400 according to this example includes a plurality of cooling packs 100 according to Example 23 and joint mechanisms 410.

FIG. 28 is a perspective view showing the outline of the cooling pack according to this example. FIG. 27 is a sectional view taken along a-a′ of FIG. 26. In the cooling unit 400, a plurality of the cooling packs 100 are filled with the above-mentioned heat storage medium, each include a heat storage layer 130 wrapped in a film 420, and are connected to each other with the joint mechanisms 410. Since the cooling unit 400 includes the joint mechanisms 410, a cooling object can be cooled with the cooling unit placed along the cooling object; hence, the cooling object can be effectively cooled.

For the purpose of increasing the strength of the cooling unit 400 or preventing the liquid leakage of the heat storage layer, a so-called pack-in-pack structure in which the outside of the film 420 is further wrapped with a film may be used.

Furthermore, the cooling unit 400 may be fixed to the cooling object in such a manner that the above-mentioned cooling unit 400 is attached to a fixing tool for fixing the cooling unit 400 to the cooling object. FIG. 28 is an illustration showing an example in which the cooling unit 400 is fixed to a human body using the fixing tool. This enables a specific portion of the human body to be effectively cooled. Examples of the fixing tool include a supporter, a towel, and a bandage.

(A) An aspect of the present invention can take an aspect below. That is, a heat storage medium according to an aspect of the present invention is a heat storage medium which undergoes a phase change at a predetermined temperature and contains water, a main agent made of a quaternary ammonium salt forming a semi-clathrate hydrate, a pH adjustor maintaining alkalinity, and a nucleating agent generating cations exhibiting positive hydration. The heat storage medium separates into a first liquid layer containing the main agent and a second liquid layer containing the nucleating agent in an environment with a temperature exceeding the phase change temperature.

Since the heat storage medium separates into the first liquid layer, which contains the main agent, and the second liquid layer, which contains the nucleating agent, in the environment with a temperature exceeding the phase change temperature as described above, the apparent concentration of the main agent can be increased and the heat storage capsule can be increased or maintained.

(B) In the heat storage medium according to the aspect of the present invention, the main agent is any one of tetrabutylammonium bromide, tetrabutylammonium chloride, tetrabutylammonium nitrate, and tetrabutylammonium fluoride; the pH adjustor is sodium carbonate; and the nucleating agent is an anhydride or hydrate of disodium hydrogen phosphate.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(C) In the heat storage medium according to the aspect of the present invention, when the main agent is tetrabutylammonium bromide, the content of tetrabutylammonium bromide is within the range of 35 wt % to 40.5 wt %, the content of the anhydride or hydrate of disodium hydrogen phosphate is 2.5 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(D) In the heat storage medium according to the aspect of the present invention, when the main agent is tetrabutylammonium bromide, the content of tetrabutylammonium bromide is within the range of 30 wt % to less than 35 wt %, the content of the anhydride or hydrate of disodium hydrogen phosphate is 3.0 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(E) In the heat storage medium according to the aspect of the present invention, when the main agent is tetrabutylammonium chloride, the content of tetrabutylammonium chloride is within the range of 29 wt % to 34 wt %, the content of the anhydride or hydrate of disodium hydrogen phosphate is 2.5 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(F) In the heat storage medium according to the aspect of the present invention, when the main agent is tetrabutylammonium chloride, the content of tetrabutylammonium chloride is within the range of 24 wt % to less than 29 wt %, the content of the anhydride or hydrate of disodium hydrogen phosphate is 3.0 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(G) In the heat storage medium according to the aspect of the present invention, when the main agent is tetrabutylammonium nitrate, the content of tetrabutylammonium nitrate is within the range of 34 wt % to 39 wt %, the content of the anhydride or hydrate of disodium hydrogen phosphate is 2.5 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(H) In the heat storage medium according to the present invention, when the main agent is tetrabutylammonium nitrate, the content of tetrabutylammonium nitrate is within the range of 29 wt % to less than 34 wt %, the content of the anhydride or hydrate of disodium hydrogen phosphate is 3.0 wt % or more, and the content of the sodium carbonate is 2.0 wt % or more.

(I) In the heat storage medium according to the present invention, when the main agent is tetrabutylammonium fluoride, the content of tetrabutylammonium fluoride is within the range of 28 wt % to 33 wt %, the content of the anhydride or hydrate of disodium hydrogen phosphate is 2.5 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(J) In the heat storage medium according to the present invention, when the main agent is tetrabutylammonium fluoride, the content of tetrabutylammonium fluoride is within the range of 23 wt % to less than 28 wt %, the content of the anhydride or hydrate of disodium hydrogen phosphate is 3.0 wt % or more, and the content of the sodium carbonate is 2.0 wt % or more.

This configuration allows the heat storage medium to undergo a phase change at a predetermined temperature and enables the heat storage medium to separate into the first liquid layer, which contains the main agent, and the second liquid layer, which contains the nucleating agent, in the environment with a temperature exceeding the phase change temperature.

(K) In the heat storage medium according to the aspect of the present invention, the specific gravity of the second liquid layer is higher than the specific gravity of the first liquid layer.

Since the specific gravity of the second liquid layer is higher than the specific gravity of the first liquid layer as described above, the heat storage medium can be separated at a temperature exceeding the phase change temperature. This enables the apparent concentration of the main agent to be increased and therefore the latent heat capacity can be increased or maintained.

(L) A cooling pack according to an aspect of the present invention is a cooling pack which controls the temperature of an article and includes the heat storage medium specified in any one of Items (A) to (K) and a housing section housing the heat storage medium.

This enables a cooling pack including a heat storage medium containing quaternary ammonium salt to be obtained, thereby enabling the temperature of an article to be controlled with high latent heat capacity.

(M) A logistics package according to an aspect of the present invention is a logistics package for packaging an article and includes a logistics package body, the cooling pack specified in Item (L), a cooling pack-holding section which is placed in the logistics package body and which holds the cooling pack, and an article-housing section which is placed in the logistics package body and which houses an article.

This enables a logistics package including a heat storage medium containing quaternary ammonium salt to be obtained, thereby enabling the temperature of an article to be controlled with high latent heat capacity even when there is a difference in temperature between the inside and outside of the logistics package in a logistics process.

(N) A cooling unit according to an aspect of the present invention is a cooling unit which cools a cooling object and includes a plurality of cooling packs which are disposed around a cooling object, which are strip-shaped, and which are specified in Item (L). The cooling packs include joint mechanisms. A plurality of the neighboring cooling packs are connected with the joint mechanisms therebetween.

This allows the cooling packs to be united with each other and also allows the cooling packs to have the degree of freedom; hence, operability upon disposing the cooling packs at the cooling object is enhanced and the cooling object can be effectively cooled.

(O) A cooling unit according to the present invention is a cooling unit which cools a cooling object and includes a plurality of cooling packs which are disposed around a cooling object, which are strip-shaped, and which are specified in Item (L) and cooling pack supports for bringing the cooling packs close to or into contact with the cooling object, each of the cooling pack supports being disposed along the periphery of a corresponding one of the cooling packs and supporting a corresponding one of the cooling packs.

This enables the cooling packs to be brought close to or into contact with the cooling object, thereby enabling the cooling object to be maintained close to the melting point of the cooling packs.

(P) In the cooling unit according to an aspect of the present invention, the cooling pack supports include joint mechanisms connecting the neighboring cooling packs.

This allows the cooling packs to be united with each other and also allows the cooling packs to have the degree of freedom; hence, operability upon disposing the cooling packs at the cooling object is enhanced.

This international application claims priority to Japanese Patent Application No. 2016-227098 filed on Nov. 22, 2016 and the entire contents of Japanese Patent Application No. 2016-227098 are incorporated by reference in this international application.

### REFERENCE SIGNS LIST

10, 10a First liquid layer
20, 20a Second liquid layer
100 Cooling pack(s)
110 Cooling pack body
120 Housing section
130 Heat storage layer
150 Heat storage medium
170 Inlet
190 Plug
200 Logistics package
210 Logistics package body
220 Cooling pack-holding section
221 Cooling pack-holding member
230 Article-housing section
240 Housing section
250 Lid section
300 Cooling unit
310 Cooling pack supports
320 Joint mechanisms
330 Fixing mechanism
400 Cooling unit
410 Joint mechanisms
420 Film

1. A heat storage medium which undergoes a phase change at a predetermined temperature, comprising:
water;
a main agent made of a quaternary ammonium salt forming a semi-clathrate hydrate;
a pH adjustor maintaining alkalinity; and
a nucleating agent generating cations exhibiting positive hydration,
the heat storage medium separating into a first liquid layer containing the main agent and a second liquid layer containing the nucleating agent in an environment with a temperature exceeding the phase change temperature.

2. The heat storage medium according to claim 1, wherein the main agent is any one of tetrabutylammonium bromide, tetrabutylammonium chloride, tetrabutylammonium nitrate, and tetrabutylammonium fluoride:
the pH adjustor is sodium carbonate; and
the nucleating agent is an anhydride or hydrate of disodium hydrogen phosphate.

3. The heat storage medium according to claim 2, wherein when the main agent is tetrabutylammonium bromide, the content of tetrabutylammonium bromide is within the range of 35 wt % to 40.5 wt %,
the content of the anhydride or the hydrate of disodium hydrogen phosphate is 2.5 wt % or more, and
the content of the sodium carbonate is 2.0 wt % or more.

4. The heat storage medium according to claim 2, wherein when the main agent is tetrabutylammonium bromide, the content of tetrabutylammonium bromide is within the range of 30 wt % to less than 35 wt %,
the content of the anhydride or the hydrate of disodium hydrogen phosphate is 3.0 wt % or more, and
the content of the sodium carbonate is 2.0 wt % or more.

5. The heat storage medium according to claim 2, wherein when the main agent is tetrabutylammonium chloride, the content of tetrabutylammonium chloride is within the range of 29 wt % to 34 wt %,
the content of the anhydride or the hydrate of disodium hydrogen phosphate is 2.5 wt % or more, and
the content of the sodium carbonate is 2.0 wt % or more.

6. The heat storage medium according to claim 2, wherein when the main agent is tetrabutylammonium chloride, the content of tetrabutylammonium chloride is within the range of 24 wt % to less than 29 wt %,
the content of the anhydride or the hydrate of disodium hydrogen phosphate is 3.0 wt % or more, and
the content of the sodium carbonate is 2.0 wt % or more.

7. The heat storage medium according to claim 2, wherein when the main agent is tetrabutylammonium nitrate, the content of tetrabutylammonium nitrate is within the range of 34 wt % to 39 wt %,
the content of the anhydride or the hydrate of disodium hydrogen phosphate is 2.5 wt % or more, and
the content of the sodium carbonate is 2.0 wt % or more.

8. The heat storage medium according to claim 2, wherein when the main agent is tetrabutylammonium nitrate, the content of tetrabutylammonium nitrate is within the range of 29 wt % to less than 34 wt %,
the content of the anhydride or the hydrate of disodium hydrogen phosphate is 3.0 wt % or more, and
the content of the sodium carbonate is 2.0 wt % or more.

9. The heat storage medium according to claim 2, wherein when the main agent is tetrabutylammonium fluoride, the content of tetrabutylammonium fluoride is within the range of 28 wt % to 33 wt %,
the content of the anhydride or the hydrate of disodium hydrogen phosphate is 2.5 wt % or more, and
the content of the sodium carbonate is 2.0 wt % or more.

10. The heat storage medium according to claim 2, wherein when the main agent is tetrabutylammonium fluoride, the content of tetrabutylammonium fluoride is within the range of 23 wt % to less than 28 wt %,
the content of the anhydride or the hydrate of disodium hydrogen phosphate is 3.0 wt % or more, and
the content of the sodium carbonate is 2.0 wt % or more.

11. The heat storage medium according to claim 1, wherein the specific gravity of the second liquid layer is higher than the specific gravity of the first liquid layer.

12. A cooling pack which controls the temperature of an article, comprising:
the heat storage medium according to claim 1; and
a housing section housing the heat storage medium.

13. A logistics package for packaging an article, comprising:
a logistics package body;
the cooling pack according to claim 12;
a cooling pack-holding section which is placed in the logistics package body and which holds the cooling pack; and
an article-housing section which is placed in the logistics package body and which houses an article.

14. A cooling unit which cools a cooling object, comprising:
a plurality of cooling packs according to claim 12, the cooling packs being disposed around a cooling object and being strip-shaped.

15. The cooling unit according to claim 14,
wherein the cooling packs include joint mechanisms and a plurality of the neighboring cooling packs are connected with the joint mechanisms therebetween.

16. The cooling unit according to claim 14, wherein the cooling pack supports for bringing the cooling packs close to or into contact with the cooling object, each of the cooling pack supports being disposed along the periphery of a corresponding one of the cooling packs and supporting a corresponding one of the cooling packs.

17. The cooling unit according to claim 14, wherein the cooling pack supports include joint mechanisms connecting the neighboring cooling packs.
